(19) United States
(12) Patent Application Publication
(10) Pub. No.: US 2019/0369119 A1
(43) Pub. Date: Dec. 5, 2019

(54) Protein Biomarkers for Identifying and Treating Aging Skin and Skin Conditions

(71) Applicant: Avon Products, Inc., Suffern, NY (US)

(72) Inventors: Yong ZHUANG, Livingston, NJ (US); Daniel THORN LEESON, Chatham, NJ (US)

(21) Appl. No.: 16/431,674

(22) Filed: Jun. 4, 2019

Related U.S. Application Data
(60) Provisional application No. 62/680,036, filed on Jun. 4, 2018.

(51) Int. Cl. G01N 33/68

(52) U.S. Cl. CPC G01N 33/6881

(57) ABSTRACT

Provided are methods of determining the aging of skin or skin disorders or conditions by measuring and evaluating levels of skin-associated biomarker proteins in a subject's skin sample. In aspects, the levels of a subset of biomarker proteins are altered, e.g., increased or decreased, in aging/aged versus non-aging/young skin. Provided are methods for more individualized and direct treatments and therapeutic options for aging skin and/or skin disorders and conditions by determining the levels of those skin biomarker proteins that correlate with aging skin and/or certain skin attributes of a subject and administering treatment products and regimens that result in altering the levels of the biomarker proteins, in particular, toward levels of the proteins in non-aged and healthy skin.

### CROSS REFERENCE TO RELATED APPLICATIONS

The present application claims priority under 35 U.S.C. § 119(e) to U.S. Provisional Application No. 62/680,036, hereby incorporated by reference in its entirety.

### FIELD OF THE INVENTION

The present invention relates to methods of identifying and diagnosing aging skin and skin conditions by assessing underlying biomolecules in skin samples. The invention also relates to methods of providing directed treatments and therapies for aging skin and skin conditions, as well as monitoring or evaluating the efficacy of such skin treatments and therapies.

### BACKGROUND

The skin is routinely subjected to numerous external and internal insults which can detrimentally affect its condition and appearance, particularly as the aging process occurs. Consumers and patients are interested in mitigating or delaying the dermatological signs of physiologically-, chronologically-, hormonally-, or photo-aged skin. During the aging process, the complexion of the skin, i.e., the color and appearance of the skin, as well as its function, deteriorate slowly from aging and/or exposure to environmental agents or conditions, such as ultraviolet radiation.

Human skin is broadly divided into three main parts: the surface or outermost epidermis which provides an anatomical and protective barrier to foreign elements and agents and which maintains the body's internal environment; the underlying dermis, composed of connective tissue and blood vessels that provide elasticity, and nutritional and structural support to the epidermis; and the subcutis, which is composed primarily of subcutaneous fat cells which function as energy deposits. The epidermis mainly consists of keratinocytes and is composed of several sublayers, including, from the innermost layer to the outermost, the stratum germinativum/stratum basale, the stratum spinosum, the stratum granulosum, and the stratum corneum.

The keratinocytes, generated by the mitosis of keratinocyte stem cells, originate in the stratum basale and then emerge through the strata. As these cells move to the surface of the skin, they undergo gradual differentiation and become enucleated, flattened and highly keratinized. During this process the keratinocytes become more organized and form desmosomes, or cellular junctions that inter-connect them. Through their production and secretion of keratin proteins and lipids, keratinocytes form an extracellular matrix which strengthens the skin. Eventually the keratinocytes die and form the stratum corneum. In healthy skin, keratinocytes are shed and replaced continuously every 30 days.

With aging and/or declining health, the dermal/epidermal junction (DEJ) flattens and reduces the amounts of nutrients/oxygen that are transferred to the epidermis through the DEJ. This is because the surface area in contact with the epidermis shrinks, and the ability of the epidermis to transfer the available nutrients/oxygen to its cellular components lessens. Inefficient nutrient/oxygen transport impacts the keratinocytes of the epidermis such that keratinocyte renewal rate is reduced. As a consequence, the stratum corneum loses its capacity to retain moisture, leading to dehydration of the skin and signs of aging, such as wrinkling and the like, which may be exacerbated by common medications or physiological events.

Methods are needed to more efficiently and effectively determine and identify a skin condition or cause(s) thereof affecting a subject so as to more efficiently and effectively provide improved treatments and therapeutic products to the subject. In addition, methods are needed to more efficiently and effectively determine and identify biomarkers of aging skin and skin conditions so as to improve the quality of aging skin and to provide better treatments and therapies. The present invention addresses such needs.

### SUMMARY OF THE INVENTION

Provided herein are methods and improved methods for determining, assessing, diagnosing, or identifying aging skin, skin conditions and the cause(s) thereof that afflict subjects by determining expression levels or levels of newly discovered skin-associated biomarkers as described herein. In embodiments, the skin-associated biomarkers are protein biomarkers. In a particular embodiment, the skin-associated biomarkers are protein biomarkers set forth in Table 3 herein, or a subset thereof. The methods are particularly advantageous for providing more directed treatments, therapies and agents that target skin conditions, including aging or aged skin, which may be correlated with underlying deficiencies or surpluses of the protein biomarkers set forth in Table 3 herein, or a subset thereof. The skin conditions, protein deficiencies or surpluses may arise during the process of aging; they may be encountered as result of a general health problem or physiological event, e.g., hormonal imbalance or changes, such as menopause; or they may be a consequence of both. It will be understood that the terms “expression levels” and “levels” are used interchangeably herein.

Another feature of the methods and improved methods described herein is the enhanced ability to treat and/or recommend treatment of a subject's skin condition, skin defect or deficiency, aging, or degree of aging more directly, effectively and efficiently, especially after determining one or more skin-associated protein biomarkers that show differential levels in a subject's skin relative to a control. In an embodiment, based on the identification or determination of differential levels of the one or more skin-associated protein biomarkers according to the described methods, the underlying causes of a skin condition or disorder may be determined or diagnosed in a subject. In another embodiment, the practice of the described methods allows for administering to the subject a directed treatment that is able to modulate the levels of the one or more skin-associated protein biomarkers so as to treat the subject's skin condition or disorder, or the underlying cause(s) thereof. It will be appreciated that the phrase “protein biomarkers” as used supra and infra embraces protein, polypeptide, peptide and proteomic biomarkers in accordance with the present invention.

Also provided are methods and improved methods which involve determining, assessing, diagnosing, or identifying levels of skin-associated protein biomarkers as described herein for determining, assessing, diagnosing, and identifying aging and age-related skin conditions and/or the effects, status, or degree thereof, in subjects and for providing more directed and effective anti-aging treatments and therapies.

The methods provided by the invention advantageously aid in the diagnosis and treatment of aging skin, or of a skin condition or deficiency, at a level that exceeds mere visual or tactile inspection by a medical practitioner, e.g., a physician or dermatologist, or a clinician. According to the present methods, the detection of levels of protein biomarkers in a sample from a subject, particularly a skin sample, may allow the determination of one or more underlying biochemical or biophysical deficiencies that causes or is associated with the skin condition or deficiency or aging, e.g., skin fragility, aberrant pigmentation, such as pigmentation loss or excess pigmentation, tendency to shear and the like. By way of nonlimiting example, the methods of the invention can detect or diagnose low levels of certain stratum corneum proteins in a subject having a certain skin condition or disorder. This type of underlying biochemical or biophysical deficiency in the subject can then be treated with a targeted therapy or agent to correct that deficiency, thus providing an advantageous diagnostic or prognostic approach for recommending or administering customized or specific targeted treatments for the skin.

The present methods also allow for the recommendation or prescribing of a treatment or therapy that is targeted to the underlying cause of the skin condition or deficiency to more directly correct or remedy it, based on the biomarker profile that is detected or assessed from a subject's skin sample. By way of example, a subject suffering from a skin deficiency or condition and/or certain signs of skin aging and requiring treatment may avoid being treated with a customary topical cosmetic or cosmeceutical product, such as a generic moisturizer or cream, and may instead be recommended or prescribed a targeted treatment to correct the skin deficiency or condition by the practice of the methods described herein. A more directed and biochemically based assessment of the subject's aging skin or skin condition via the present methods could advantageously deliver better results for treating the subject.

The methods described herein provide improved treatments and therapeutics for a subject who suffers from aging skin and/or who is diagnosed with a skin condition, disease, or disorder. Following assessment of the subject's skin sample to determine a skin-associated protein biomarker expression profile by the practice of the methods of the present invention, specific or customized skin treatments, compositions and products that produce therapeutic effects, optimally without undesirable side effects, can be recommended and/or administered to the subject. In an embodiment, certain proteins, such as one or more of the proteins presented in Table 3, increase or show a trend of increasing with age or in aging skin versus non-aging or young skin, and/or in association with a skin attribute, e.g., wrinkling or pigmentation. In another embodiment, certain proteins, such as one or more of the proteins presented in Table 3, decrease or show a trend of decreasing with age or in aging skin versus non-aging or young skin, and/or in association with a skin attribute, e.g., wrinkling or pigmentation. In embodiments, examples of proteins whose levels may decrease with age include the heat shock proteins HSPA9, HSPA5 and HSPA8, which may thus contribute to a look and feel of aging skin in a subject. Accordingly, the assessment of an increased fold change in levels of one or more of these proteins, or other proteins in Table 3, in the control (e.g., young, non-aged skin) versus levels in the skin of a subject undergoing testing may be indicative, or even predictive, of aging or aged skin. Treatments to increase the levels of one or more of these proteins, such as toward levels that are found in young, non-aging or non-aged skin, would be desirable to treat, counteract, reduce, or ameliorate the aging of a subject's skin.

In a particular embodiment, the methods described herein provide improved treatments and therapeutics for a subject who has aging or aged skin. The surface of aging or aged skin may exhibit lines and wrinkles, creases, sagging, dullness, discoloration, uneven tone, rough texture, and the like. In addition, aging or aged skin exhibits less strength and flexibility and is more fragile. Following assessment of the protein expression profiles of a subject's skin sample by the practice of the described methods, certain or customized products that retard or counter the aging effects on the skin may be administered or provided to the subject. Provided are improved methods to address the restoration of skin texture and the reduction of wrinkling, fine lines and inflammation associated with aging or aged skin by assessing levels of skin-associated biomarker proteins to identify underlying cause(s), which can result in more targeted and useful treatment regimens for counteracting or retarding skin aging (or degree or extent of aging), particularly when the levels of certain protein biomarkers are assessed and determined from a skin sample relative to suitable controls.

Provided by the present invention are methods for assessing skin conditions or aging skin using quantitative proteomics analyses. The methods relate to the diagnosis or determination of skin conditions or factors involved in aging or aged skin versus young (non-aging or non-aged) skin by assessing changes or alterations, e.g., decreases or increases, in the levels of one or more skin-associated protein biomarkers in skin samples obtained from a subject undergoing assessment compared with a suitable control. In an embodiment, the one or more protein biomarkers are as set forth in Table 3 herein. In an embodiment, the one or more protein biomarkers are a subset or panel of the proteins presented in Table 3 herein. In a particular embodiment, the one or more protein biomarkers are heat shock proteins (HSP), or HSP70 proteins, including one or more of HSPA9, HSPA5 and HSPA8.

In an embodiment, the subject undergoing assessment exhibits a skin condition or disease and the control does not. In an embodiment, the subject undergoing assessment has aged or aging skin and the control is a younger subject who does not have aged or aging skin. In embodiments, the control is a healthy individual without aging skin or signs thereof. In embodiments, the control is a non-aged or aging skin sample obtained from the same subject as the aged or aging skin sample. In an embodiment, the change in the levels of the one or more skin-associated protein biomarkers in a suitable control versus the subject undergoing testing is a fold-increase, such as shown in Table 3 herein, for example, an increase of about or equal to 1.5 fold or greater, such as 1.6 fold, 1.7 fold, 1.72 fold, 1.73 fold, 1.74 fold, 1.75 fold, 1.76 fold, 1.77 fold, 1.78 fold, 1.79 fold, 1.8 fold, 1.9 fold, 2 fold, 3 fold, 4 fold, 4.1 fold, 4.2 fold, 4.3 fold, 4.4 fold, 4.5 fold, 4.6 fold, 4.7 fold, 4.8 fold, 4.9 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, or 20 fold, or greater, as well as values and sub-values between the listed values. In an embodiment, the change in the levels of the one or more skin-associated protein biomarkers in a suitable control versus the subject undergoing testing is a fold-decrease, such as shown in Table 3 herein, for example, a decrease of about or equal to 1.2 fold, 1.21 fold, 1.23 fold, 1.24 fold, 1.25 fold, 1.26 fold, 1.27 fold, 1.28 fold, 1.29 fold, 1.3 fold, 1.31 fold, 1.32 fold, 1.33 fold, 1.34 fold, 1.35 fold, 1.36 fold, 1.37 fold, 1.38 fold, 1.39 fold, 1.4 fold, 1.41 fold, 1.42 fold, 1.43 fold, 1.44 fold, 1.45 fold, 1.46 fold, 1.47 fold, 1.48 fold, 1.49 fold, 1.5 fold, 1.51 fold, 1.52 fold, 1.53 fold, 1.54 fold, 1.55 fold, 1.56 fold, 1.57 fold, 1.58 fold, 1.59 fold, 1.6 fold, 1.61 fold, 1.62 fold, 1.63 fold, 1.64 fold, 1.65 fold, 1.66 fold, 1.67 fold, 1.68 fold, 1.69 fold, 1.7 fold, 1.71 fold, 1.72 fold, 1.73 fold, 1.74 fold, 1.75 fold, 1.76 fold, 1.77 fold, 1.78 fold, 1.79 fold, 1.8 fold, 1.81 fold, 1.82 fold, 1.83 fold, 1.84 fold, 1.85 fold, 1.86 fold, 1.87 fold, 1.88 fold, 1.89 fold, 1.9 fold, 1.95 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.57 fold, 3.6 fold, 3.67 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, 12 fold, or 12.5 fold, as well as values and sub-values between the listed values. In an embodiment, altered levels of one or more heat shock proteins (HSPs), e.g., one or more of HSPA9, HSPA5 and HSPA8, detected in a subject's skin sample according to the methods of the invention may be modulated by administering to the subject an effective treatment comprising a treatment or therapeutic product or regimen that is capable of increasing the levels of these HSPs in a skin sample identified as having deficient levels, e.g., an aging or aged skin sample, versus levels in a control, e.g., a young, non-aged skin sample. Illustratively and without limitation, examples of compounds and agents that induce HSP70 proteins include shikonin, Arnica montana, resveratrol and green tea polyphenol. (See, e.g., FIG. 3).

In an embodiment, the protein biomarker expression profile determined from a subject's skin sample according to the methods of the invention is predictive of the age or aging of the subject. The levels of one or more of the protein biomarkers assessed in a skin sample are predictive of and correlated with the aging process. In an embodiment, levels of one or more of the protein biomarkers assessed in a skin sample are predictive of and correlated with how well a person is aging, based on changes or alterations in the levels of one or more of such proteins over time. In an embodiment, the levels of certain biomarker proteins assessed from a subject's skin sample are indicative of whether the subject is “aging gracefully” or whether the subject shows premature or excessive aging, without limitation to any particular cause. The methods of the invention serve to correlate one or more overt or underlying skin characteristics or conditions with changes in the levels of one or more, or two or more, or three or more, or four or more, etc., proteins biomarkers as presented in Table 3. By way of example, skin wrinkles or pigmentation aberrations or alterations, e.g., discoloration, hyperpigmentation, or hypopigmentation, and the like, may be caused by deficiencies in one or more of the protein biomarkers according to the invention, which can be assessed or determined by the methods of the present invention. By way of further example, skin wrinkles or pigmentation aberrations or alterations, e.g., discoloration, hyperpigmentation, or hypopigmentation, and the like, may be caused by increased levels of one or more of the protein biomarkers, which can be assessed or determined by the methods of the present invention. In an embodiment, the levels of the one or more protein biomarkers of Table 3 may decrease over time and correlate with or trend toward increased aging of the subject's skin. In an embodiment, the levels of the one or more protein biomarkers of Table 3 may increase over time and correlate with or trend toward increased aging of the subject's skin. Once the underlying protein deficiency(ies) or increases are assessed or determined by the practice of the present methods, an optimal and appropriate treatment regimen or therapeutic may be determined and administered to the subject.

In another embodiment, a protein biomarker expression profile determined from a subject's skin sample according to the methods of the invention demonstrates the strength or weakness, e.g., fragility, lack of elasticity, of the subject's skin, such that a customized treatment can be provided to specifically address, e.g., reduce or counteract, the weakness. For example, if a subject is found to have low levels of, or to be deficient in, one or more of the protein biomarkers of Table 3 relative to control levels, following analysis of the subject's skin sample, a treatment regimen that focuses on altering or modulating the deficiency may be undertaken. In an embodiment, the treatment regimen focuses on increasing the levels of the one or more protein biomarkers of Table 3 that are deficient in a subject's sample relative to control levels. In another embodiment, the treatment regimen focuses on decreasing the levels of the one or more protein biomarkers of Table 3 that are overexpressed or increased in a subject's sample relative to control levels. In a particular embodiment, a treatment regimen is designed to increase the levels of one or more of the proteins HSPA9, HSPA5 and HSPA8.

The present methods allow the clinician, medical practitioner, and even the subject her/himself to determine and understand that a “common” skin disorder or condition, such as wrinkles or facial lines, for example, may be based on a certain protein biomarker profile or certain protein levels that differ from those of another individual who also has wrinkles. In this way, the methods of the invention allow for individualized treatment of subjects having skin wrinkles depending on the levels of certain of the underlying protein biomarkers of Table 3 herein.

The present invention also provides methods for the screening, identification, and selection of candidate compounds, agents and drugs for treating a skin condition or a particular skin attribute, particularly, an adverse skin attribute, in a subject, including age/aging-related and health-related skin conditions, by determining from a skin sample obtained from a subject having a skin condition, such as wrinkles, lines, rhytids, rash, acne, etc., the levels of one or more of the protein biomarkers in Table 3 or a subset of the biomarker proteins from Table 3 that correlate with the particular skin condition or attribute. A candidate compound, agent or drug is contacted with a skin sample obtained from a subject having the skin condition under suitable conditions for a predetermined time period, and the skin sample that had contacted the candidate compound, agent or drug is assessed for modulated levels of one or more of the protein biomarkers in Table 3 or a subset of the biomarker proteins from Table 3, and the results are compared with those of a skin sample prior to contact with, or a sample naïve to contact with, the candidate compound, agent or drug. Modulation of the levels of one or more proteins of Table 3 may be observed as a decrease or an increase in levels following contact with the candidate compound, agent, or drug. In related embodiments, the present methods are useful for identifying substances (compounds, agents, or drugs) that are effective in treating or preventing the aging of skin, in which a substance can decrease or increase the levels of the protein biomarkers as set forth in Table 3 herein, or modulate a profile of the protein biomarkers in Table 3, correlated with aged or aging skin. In an embodiment, the method identifies a substance that decreases the levels of one or more proteins of Table 3. In an embodiment, the method identifies a substance that increases the levels of one or more skin protein biomarkers of Table 3.

In embodiments, the methods of the invention allow for the monitoring of changes in the skin status of a subject over time, in particular, at the start of, during and/or after the course of a medical or cosmeceutical treatment to address a skin condition related to health or aging, by virtue of testing skin samples of the subject at predetermined time periods during or after treatment to assess whether any changes in the levels of the protein biomarkers are evident. For example, such monitoring could assess and determine whether deficiency(ies) in one or more of the protein biomarkers in the subject's skin sample had changed, improved, attained normal or close-to-normal levels, and the like, as a consequence of the administration of a medical or cosmeceutical treatment or therapeutic.

In an aspect, the invention provides a method of assessing the degree of skin aging or of an age-associated skin attribute in a subject, which involves (i) assaying in a skin sample obtained from the subject the levels of a subset of skin-associated biomarkers set forth in Table 3 relative to control levels; (ii) measuring an alteration in the levels of subset of skin biomarkers set forth in Table 3 relative to control levels; and (iii) assessing degree of skin aging or an age-associated skin attribute in the subject based on the measured alteration comprising increased or decreased levels of the subset of skin biomarkers in Table 3 relative to the control levels. In an embodiment, the subset of skin-associated biomarkers are one or more or two or more of the proteins set forth in Table 3. In an embodiment, the alteration measured comprises an increased fold change in levels of one or more of the following skin-associated biomarker proteins in the control (e.g., young, non-aged skin) relative to levels in the skin of a subject undergoing testing: GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD set forth in Table 3, wherein a deficiency in the levels of the skin-associated biomarker proteins, or a subset thereof, in the subject's skin indicates skin aging in the subject. In an embodiment, the method further involves administering to the subject a treatment regimen or therapeutic product which causes an increase in the levels of one or more of the skin-associated biomarker proteins in the skin of the subject, thereby treating aging skin and/or an associated skin attribute, e.g., wrinkling or pigmentation, in the subject. In another embodiment of the method, the alteration measured comprises a decreased fold change in levels of one or more of the following skin-associated biomarker proteins in the control (e.g., young, non-aged skin) versus levels in the skin of a subject undergoing testing: TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB set forth in Table 3 in the skin of the subject relative to control levels, and wherein an increase in the levels of the subset of skin-associated biomarker proteins in the subject's skin indicates skin aging in the subject. In another embodiment, the method further comprises administering to the subject a treatment regimen or therapeutic product which causes an increase in the levels of one or more of the skin-associated biomarker proteins in the skin of the subject, thereby treating aging skin in the subject. In an embodiment, the subset of skin-associated biomarkers comprises heat shock proteins (HSPs) set forth in Table 3, in particular, HSPA9, HSPA5 and HSPA8. In an embodiment of the above methods, if an increase in the levels of the one or more biomarkers of Table 3 is identified in an aging subject's skin sample, a treatment which decreases the levels of the biomarkers toward control levels is administered to the subject or is recommended for administration to the subject to treat or prevent the subject's skin aging or attribute. In another embodiment of the above methods, if a decrease in the levels of the one or more biomarkers of Table 3 is identified in an aging subject's skin sample, a treatment which increases the levels of the biomarkers toward control levels is administered to the subject or is recommended for administration to the subject to treat or prevent the subject's skin aging or attribute. In an embodiment, the skin sample is a stratum corneum sample. In an embodiment, the skin sample is from a subject of 50 to 60-plus years of age and the control comprises subjects of 18 to 20 years of age. In an embodiment, skin aging or the skin attribute comprises one or more of wrinkles, fine lines, creases, folds, sagging, fragility, aberrant pigmentation, dull complexion, or weakness.

In another aspect, the invention provides a method of treating a skin condition or disorder in a subject, which involves (a) measuring the levels of one or more skin biomarker proteins of Table 3 in a skin sample obtained from the subject; (b) determining the levels of the one or more skin biomarker proteins in step (a) relative to levels of the same protein biomarkers in a skin sample from control subjects; wherein increased or decreased levels of the one or more skin biomarker proteins relative to the control levels identifies the skin condition or disorder in the subject; and (c) administering to the subject a treatment that targets the skin condition or disorder associated with the increased or decreased levels of the expressed skin biomarker proteins, thereby treating the subject's skin condition or disorder. In an embodiment, the skin sample is a stratum corneum sample. In an embodiment, the treatment comprises increasing the levels of one or more of a subject's skin-associated biomarker proteins comprising GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD as set forth in Table 3 toward control levels, thereby treating the skin condition or disorder in the subject. In an embodiment, the treatment comprises increasing the levels of one or more of HSPA9, HSPA5 and HSPA8 in the subject's skin. In another embodiment, the treatment comprises decreasing the levels of one or more of a subject's skin-associated biomarker proteins comprising TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB as set forth in Table 3 toward control levels, thereby treating the skin condition or disorder in the subject. In an embodiment, the subject's skin condition or disorder comprises wrinkles and lines in the skin. In embodiments, the subject is 50 to 60-plus years of age, and the control is a young subject having healthy and normal skin in the absence of the skin condition or disorder.

In another aspect, the invention provides a method of predicting the likelihood that a subject has, or is at risk for developing, an adverse skin attribute or aging skin, in which the method involves (a) measuring the levels of subset of protein biomarkers from Table 3 in a skin sample obtained from the subject; (b) identifying the levels of the subset of protein biomarkers in step (a) relative to levels from a suitable control; and (c) predicting that subject has, or is at risk of developing, an adverse skin attribute or aging skin by assessing increased or decreased levels of the subset of protein biomarkers relative to the control, which increased or decreased levels correlate with the adverse skin attribute or aging skin. In an embodiment, the adverse skin attribute is selected from aging skin, mottled pigmentation, wrinkles, or a combination thereof, in the skin. In an embodiment, a decreased fold change in levels of one or more of the subset of protein biomarkers TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB as set forth in Table 3, in the control (e.g., young, non-aged skin) relative to levels in the skin of a subject undergoing testing may correlate with the adverse skin attribute or skin aging. In another embodiment, an increased fold change in levels of one or more of the subset of protein biomarkers of the subset of protein biomarkers GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD as set forth in Table 3 in the control (e.g., young, non-aged skin) versus levels in the skin of a subject undergoing testing may correlate with the adverse skin attribute or skin aging. In an embodiment, the subset of protein biomarkers of Table 3 whose levels may correlate with aging or aged skin comprises heat shock proteins (HSPs) HSPA9, HSPA5 and HSPA8, and, in particular, HSPA5.

In another of its aspects, the invention provides a method of treating aging skin in a subject in need thereof, in which the method involves (a) measuring the levels of a subset of protein biomarkers in Table 3 in a skin sample obtained from the subject; (b) identifying the levels of the subset of protein biomarkers in step (a) relative to levels of the same proteins in a skin sample from control subjects; wherein decreased levels of the subset of protein biomarkers relative to control levels identifies aging skin in the subject; and (c) administering to the subject identified as having aging skin a treatment that increases the levels of the subset of protein biomarkers in the subject's skin, thereby treating aging skin in the subject. In an embodiment, the subject in need is 50 to 60-plus years of age and the control comprises subjects 18-20 years of age. In an embodiment, the subset of the protein biomarkers in Table 3 which decrease or trend toward decreasing in aging or aged skin comprises one or more of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD, and, in particular, the HSPs HSPA9, HSPA5 and HSPA8.

In another aspect, the present invention provides a method of treating aging skin in a subject in need thereof, in which the method involves (a) measuring the levels of a subset of protein biomarkers in Table 3 in a skin sample obtained from the subject; (b) identifying the levels of the subset of protein biomarkers in step (a) relative to levels of the same proteins in a skin sample from control subjects; wherein increased levels of the subset of protein biomarkers relative to control levels identifies aging skin in the subject; and (c) administering to the subject identified as having aging skin in step (b) a treatment that decreases the levels of the subset of protein biomarkers in the subject's skin, thereby treating aging skin in the subject. In an embodiment, the subject in need is 60-plus years of age and the controls comprise subjects 18-20 years of age. In an embodiment, the subset of the protein biomarkers in Table 3 which increase or trend toward increasing in aging or aged skin comprises one or more of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

In another of its aspects, the present invention provides a method of determining a treatment for aging or aged skin or a symptom thereof in a subject in need thereof, in which the method involves assaying in a skin sample obtained from the subject the levels of a subset of the skin biomarkers set forth in Table 3 relative to control levels; and administering to the subject a treatment that changes the levels of the subset of the biomarkers in Table 3 associated with aging or aged skin toward levels of the same subset of biomarkers in skin of controls, if an increase or a decrease in the assayed levels of the subset of skin biomarkers in Table 3 relative to the control levels indicates aging or aged skin or a symptom thereof in the subject.

In another aspect, the invention provides a method of identifying a substance that modulates biomarkers associated with skin aging and/or a skin attribute, in which the method comprises: contacting a skin sample to be tested with a candidate substance under suitable conditions for a predetermined period of time; quantifying the levels of a subset of protein biomarkers of Table 3 from the contacted skin sample, wherein levels of the subset of proteins change with skin aging and/or a skin attribute; determining the levels of the proteins of the contacted skin sample relative to the levels of a control to evaluate the effect of the substance on the levels of the subset of proteins from the sample; and identifying the substance that modulates the levels of the biomarkers associated with skin aging and/or the skin attribute in the skin sample relative to the control. In an embodiment of the method, the skin aging and/or a skin attribute comprises wrinkles, lines, creases, abnormal pigmentation, sagging, or weakness of the skin. In an embodiment of the method, the subset of proteins comprises one or more of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD. In an embodiment of the method, the subset of proteins comprises one or more of HSPA9, HSPA5 and HSPA8. In an embodiment of the method, the levels of the subset of proteins decrease with aging skin. In an embodiment of the method, the subset of proteins comprises one or more of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB. In an embodiment of the method, the levels of the subset of proteins increase with aging skin.

In an embodiment of any of the above aspects, a particular protein biomarker identified (and quantified) in skin is considered to correlate positively with a skin condition, e.g., wrinkles or fine lines, if a higher level of the protein biomarker relative to a control level is found in a skin sample of a subject having the skin condition, or more of the skin condition, or a greater degree or extent of the skin condition, e.g., wrinkles or fine lines. If a positive correlation exists, the risk of a subject developing more wrinkles or fine lines can be predicted by determining the presence and level of the protein biomarker in the subject's skin. In another embodiment of any of the above aspects, the positive correlation of a specific protein biomarker, or level thereof, with a certain skin condition, such as wrinkles or fine lines, indicates that a higher level of the protein correlates with more (a greater amount) of the wrinkles or fine lines; consequently, an anti-aging treatment or regimen can be provided (administered) to reduce specifically the level of the specific protein in the skin so as to lead to the reduction in wrinkles in the subject's skin.

In an aspect, the present invention provides a kit for determining skin aging or skin attribute status in a human subject, in which the kit comprises (a) a substrate for collecting a skin sample from the patient; and (b) a means for measuring the levels of the protein biomarkers of Table 3 relative to control levels. In an embodiment, the kit comprises a means for measuring the levels of a subset of protein biomarkers selected from the group consisting of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD, and in particular, the subset of protein biomarkers including HSPA9, HSPA5 and HSPA8. In another embodiment, the kit comprises a means for measuring the levels of a subset of protein biomarkers selected from the group consisting of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

In an aspect, the present invention provides a kit for determining skin aging or skin attribute status in a human subject. In an embodiment, the kit comprises (a) a substrate for collecting a skin sample from the subject; and (a) a means for quantifying the levels of the protein biomarkers of Table 3 relative to control levels. In an embodiment, the kit comprises (a) a substrate for collecting a skin sample from the subject; (b) a means for extracting one or more protein biomarkers of Table 3 from said substrate; and (c) a means for analyzing and/or quantifying the levels of the one or more extracted protein biomarkers relative to control levels. In embodiments, the analysis and/or quantification may be performed at point of service or at another, off-site location, especially if the sample is appropriately stored, e.g., at −20° C. or −80° C. In an embodiment, the one or more protein biomarkers comprise a subset of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD, and in particular, the subset of protein biomarkers including HSPA9, HSPA5 and HSPA8. In another embodiment, the one or more protein biomarkers comprise a subset of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

### DESCRIPTION OF THE FIGURES

FIG. 1 depicts anatomical schematics of the tape stripping (D-SQUAME™) areas on the skin sites evaluated in the clinical study described in Example 1 herein. Shown on the anatomical schematics are locations of the measurement sites and the areas (sub-sites) for D-SQUAME™ strippings on the left (L) dorsal forearm, left upper-inner arm and the face. In FIG. 1, five D-SQUAMES were taken from each D-SQUAME area, for a total of 60 D-SQUAMES.

FIGS. 2A-2C presents bar graphs showing correlations between the levels of various protein biomarkers and self-perception of wrinkles as described in Example 2. FIG. 2A: detection of HSPA5, a member of the HSP70 protein family, in a subject's skin sample is observed to correlate with the subject's self-perception of wrinkles (P<0.05). FIG. 2B: detection of CAPZB (F-actin-capping protein subunit beta), an actin capping protein that is involved in growth regulation of the actin filament, in a subject's skin sample is observed to correlate with the subject's self-perception of wrinkles (P<0.05). FIG. 2C: detection of IL1RA/1A ratio (Interleukin 1 Receptor Antagonist: Interleukin 1alpha ratio), in a subject's skin sample is observed to correlate with the subject's self-perception of skin sensitivity (P<0.00845). An increased IL1RA/1A ratio in the stratum corneum has been reported to reflect potential process of skin regulation against inflammation (Terui, T. et al., 1998, Exp. Dermatol., 7(6):327-334).

FIG. 3 presents bar graphs showing that a number of different agents/compounds were found to induce HSP70 levels in skin (dermal fibroblasts). Normal human dermal fibroblasts were cultured in 6-well tissue culture treated plates. Cells were treated with test material (agent/compound) at the concentrations indicated, or with respective vehicle control diluted in growth medium, for 24 hours in a humidified 37° C. incubator with 5% CO2. After incubation, growth medium from each plate was removed and cells were lysed. HSP70 protein level in the cells was determined by a commercially available ELISA kit (Abcam, Cambridge, Mass.). In FIG. 3, Control induction value=1; shikonin (1 μM concentration) induction value=1.7; Arnica Montana (0.003% concentration) induction value=1.86; resveratrol (0.003% concentration) induction value=1.47; and G. T. polyphenol (0.003% concentration) induction value=1.24.

FIG. 4 shows the fold change in protein levels of human dermal fibroblasts following administration of the indicated agent. Results are shown as compared to control and samples labeled with “*” indicate a p value compared to the control of less than 0.05.

FIG. 5 shows the fold change in protein levels of human dermal fibroblasts following administration of the indicated agent. Results are shown as compared to control and samples labeled with “*” indicate a p value compared to the control of less than 0.05.

FIG. 6 shows the fold change in protein levels lysed 3D human skin cultures following administration of the indicated agent. Results are shown as compared to control and samples labeled with “*” indicate a p value compared to the control of less than 0.05.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, drug, antibody, nucleic acid molecule, or polypeptide, or fragments thereof.

By “alteration” or “modulation” is meant an increase or decrease relative or compared to a suitable control. An alteration or modulation may be an increase or decrease relative to a control by as little as 0.5%, 1%, 2%, 3%, 4%, 5%, 10%, 15%, 20%, 25%, 30%, 35%, or by 40%, 50%, 60%, 70%, or even by as much as 75%, 80%, 85%, 90%, 95%, or 100%, as well as percentages there between.

By “antibody” is meant any immunoglobulin polypeptide, or a fragment thereof, that binds to an antigen or immunogen.

The term “skin status” refers to the status or condition of the skin of a subject or patient. Examples of types of skin status include, but are not limited to, the subject's age, the subject's risk of skin disease, the presence or absence of one or more symptoms of skin aging and/or a skin disorder, the presence or absence of a skin disorder or disease, the stage of aging or skin disease in a subject or patient, and the effectiveness of treatment of the skin disease or aging of skin, e.g., counteracting, reducing, improving, or eradicating skin aging or disease.

The terms “aging”, aged”, or “old” refer to adult individuals who are typically over 40 years of age or greater, or over 50 years of age or greater, or over 60 years of age or greater, or 65 years of age or greater (“65+”), or 70 years of age or greater, or 80 years of age or greater, or 90 years of age or greater. In certain embodiments, the individual or subject is at least 50 years of age or older. In other embodiments, the individual or subject is at least 60 years of age or older (i.e., “60-plus” years of age). It will be understood that certain skin conditions, aging skin, or symptoms thereof may also result from certain genetic or medical diseases, disorders, or syndromes that affect a child or a young, non-adult individual who is under 20 or 30 years of age. For example, premature aging syndromes, also known as progeria, are known to include two, rare, inherited conditions. Hutchinson-Gilford syndrome and Werner syndrome (also known as adult progeria). Both conditions are characterized by skin changes that indicate premature aging. In addition, depending on one's level of exposure to sunlight or other environmental factors, individuals who are 40 years old or younger may experience skin conditions and aging skin that are more typically found to affect older individuals. The methods of the invention are suitable for all such individuals who experience aging skin symptoms and/or a skin disorder or disease.

The terms “non-aging”, “non-aged”, or “young,” refer to individuals who are typically in the range of 15 to 25 years of age, or 18 to 25 years of age, or 18 to 20 years of age. Individuals in the age range of about 18 to about 20 years may serve as a control group for those who are in an aged group.

By “biologic sample” or “patient sample” is meant any tissue, cell, fluid, skin, or other material derived from a subject or organism, preferably, a mammalian subject or organism, particularly a human subject or patient. For example, tissue samples include skin samples and biopsy samples, such as tissue or skin biopsy samples, tissue cell samples and skin cell samples, e.g., skin fibroblast cells. Bodily fluids include, but are not limited to, blood, blood serum, plasma, saliva, urine, peritoneal fluid, ascites, pleural effusions, tears, and fluids from cysts of the skin. In a particular embodiment of the described methods, a subject's biologic sample is a skin sample. In embodiments, the skin sample is obtained from the face of a subject, particularly a human subject. In embodiments, the skin sample may be obtained from any area of a subject's body and its dorsal and ventral surfaces, including the head, face, neck, torso, trunk, pelvis, thighs, arms, hands, fingers, legs, knees, feet, toes, and buttocks.

By “capture molecule” or “capture reagent” is meant any polypeptide or polynucleotide capable of specifically binding a polypeptide of interest. In one embodiment, a capture molecule is an antibody the specifically binds a polypeptide or protein biomarker of interest.

As used herein, the terms “determining”, “identifying”, “assessing”, “assaying”, “measuring”, “quantifying”, and “detecting” refer to both quantitative and qualitative determinations, and as such, the terms “determining” and “detecting” are used interchangeably herein with “assaying”, “measuring”, and the like.

By “reference” or “control” is meant a standard of comparison. For example, the levels of protein, polypeptide, proteomic, or polynucleotide biomarkers present in a patient sample, such as a skin sample, may be compared to the levels of the protein, polypeptide, proteomic, or polynucleotide present in a corresponding healthy cell or tissue or skin reference or control and/or present in a young, non-aged control. The control or control skin is used to establish a standard of comparison for determining or ascertaining experimental effects and/or the degree or extent of variation of effects that occur during and/or result from the study. With particular regard to the described methods, a control for aging or aged skin is skin from a young, non-aged subject, e.g., of about 18 to about 25 years of age, or of about 18 to 20 years of age. The skin of subjects younger than 18 years is also contemplated for use as a control for aging skin or a skin condition or disorder.

By “periodic” is meant at regular intervals. Periodic patient monitoring includes, for example, a schedule of tests that are conducted, performed, or administered daily, bi-weekly, bi-monthly, monthly, bi-annually, or annually.

A “biomarker” or “marker” as used herein generally refers to a protein, polypeptide, peptide, nucleic acid, clinical indicator molecule or other agent that is associated with a skin condition, e.g., aging skin, or skin disease. In one embodiment, a marker of aging or aged skin is a protein, glycoprotein, polypeptide, or peptide that is differentially expressed or present (or whose levels are differentially expressed or present) in a biological sample obtained from an aged or older subject relative to a control or reference. In another embodiment, a marker of a skin condition, disorder, or disease is a protein, polypeptide, or peptide that is differentially expressed or present (or whose levels are differentially expressed or present) in a biological sample obtained from a subject having or at risk of developing a skin condition or disease relative to a control or reference. In a particular embodiment, a marker of aging skin or of a skin condition or disease is a protein that is differentially present (or whose level is differentially expressed) in a biological sample obtained from an aged or aging subject, or from a subject having or at risk of developing a skin condition or disease relative to a suitable control or reference. A marker is differentially present if the mean or median level of the marker present in the sample is statistically different from the level present in a reference. A reference level may be, for example, the level present in a sample obtained from a non-aged (young) control subject, or from a healthy or “normal” control subject, e.g., a subject not having the skin condition or disease, or the level obtained from the subject at an earlier time point, i.e., prior to aging, assessment, or treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. Markers useful according to the methods of the invention include, for example, the proteins set forth in Table 3 herein, or a subset thereof. Fragments useful in the methods of the invention are sufficient to bind an antibody that specifically recognizes the protein from which the fragment is derived. The invention includes markers that are substantially identical to the amino acid sequence or encoding nucleic acid sequence of a biomarker protein or polypeptide or peptide. Preferably, such a sequence is at least 85%, 90%, 95% or even 99% identical at the amino acid level or nucleic acid to the sequence used for comparison.

By “marker” or “biomarker profile” is meant a characterization of the expression or levels of one or more, or a subset of, polypeptides or polynucleotides. In an embodiment, a protein biomarker profile reflects the levels of two or more proteins of Table 3 herein. By way of nonlimiting example, the levels of one or more of the protein biomarkers of Table 3 herein provide a marker or biomarker profile associated with a given subject's or patient's skin aging status, degree, or process, or skin disorder, condition, or disease.

“Modulate” as used herein refers to changing (e.g., increasing, over-expressing, decreasing, reducing, or eliminating), modifying, or regulating the level or amount, (or expression, activity, or function) of one or more protein biomarkers or biomarker profiles as described herein. Such modulation may occur through the effects of one or more agents or compounds that induce increased or decreased levels of a target molecule in skin. In an embodiment, modulating refers to increasing or decreasing a level or amount. In an embodiment, a substance (compound, small molecule, agent, or drug), i.e., candidate substance, can be effective in treating or preventing the aging of skin, or an aging skin phenotype, in which the substance can modulate, e.g. decrease or increase, the levels of one or more proteins of Table 3, or protein biomarker profiles, correlated with aged or aging skin.

A “skin attribute” as used herein refers to a physical feature that is characteristic of aging skin and/or a skin condition in a subject. In embodiments, a skin attribute may be an feature, or an adverse feature, of aging skin or a skin condition. By way of example, skin attributes that are characteristic of aging include, without limitation, wrinkles or wrinkling, lines, creases, folds, sagging, stretching, weakness, dryness, dullness, flakiness, discoloration and abnormal pigmentation such as hypo- or hyper-pigmentation. In embodiments, a subject may exhibit attributes of both aging skin and a skin condition or disorder having the same or similar characteristics.

By “immunological assay” is meant an assay that relies on an immunological reaction, for example, an antibody binding to an antigen. Examples of immunological assays include ELISAs, Western blots, immunoprecipitations, protein or nucleic acid microarray or macroarray and other assays known to the skilled practitioner. “Microarray” means a collection of proteins, polypeptides, peptides, or nucleic acid molecules from one or more organisms arranged on a solid support (for example, a chip, plate, or bead). These proteins, polypeptides, peptides, or nucleic acid molecules may be arranged in a grid where the location of each protein, polypeptide, peptide, or nucleic acid molecule remains fixed to aid in identification of the individual proteins, polypeptides, peptides, or nucleic acid molecules, e.g., from a sample of a subject or patient undergoing testing, assessment, or analysis.

By “multiplex assay” is meant an assay where two or more agents or analytes are detected concurrently.

By “panel” or “subset” is meant a collection of molecules, e.g., protein, polypeptide, peptide, or nucleic acid. In a particular embodiment, the panel or subset of molecules encompass proteins, polypeptides, or peptides, such as those described herein. If desired, the panel or subset is fixed to a solid substrate or support. In embodiments, a panel or subset of protein molecules is detected from a larger population of protein biomarkers, based on their levels in skin, as determined by the practice of the described methods.

The term “subject” or “patient” or “individual” refers to an animal which is the object of, or candidate for, treatment, observation, assessment, or experiment. By way of example only, a subject refers to a mammalian animal (a mammal), including, but not limited to, a human, a non-human primate, or a non-human mammal such as a murine, bovine, equine, canine, ovine, or feline mammal. The terms “subject,” “patient,” and “individual” are used interchangeably herein. In preferred embodiments, the subject is a human being. Both male and female subjects are embraced by the described methods.

By “specifically binds” is meant recognition and binding of a molecule (e.g., polypeptide, peptide, or protein), ligand (e.g., a cognate ligand) by a compound or molecule (e.g., antibody) that does not substantially recognize and bind, or nonspecifically bind, other molecules in a sample, e.g., a biological sample.

The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cut-off points of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the condition or disease in question. An area under the curve (referred to as “AUC”) of 1 represents a perfect test. In embodiments, biomarkers and diagnostic methods of the present invention have an AUC greater than 0.50, greater than 0.60, greater than 0.70, greater than 0.80, or greater than 0.9. Other useful measures of the utility of a test are positive predictive value (“PPV”) and negative predictive value (“NPV”). PPV is the percentage of actual positives that test as positive. NPV is the percentage of actual negatives that test as negative.

Unless specifically stated or obvious from context, as used herein, the term “about” is understood as being within a range of normal tolerance in the art, for example and without limitation, within 2 standard deviations of the mean. About can be understood to be within 10%, 9%, 8%, 7%, 6%, 5%, 4%, 3%, 2%, 1%, 0.5%, 0.1%, 0.05%, or 0.01% of the stated value. Unless otherwise clear from context, numerical values provided herein are modified by the term about.

Ranges provided herein are understood to be shorthand for all of the values within the range, or between the stated values of the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural. Thus, for example, reference to “a biomarker” includes reference to more than one biomarker.

The term “including” is used herein to mean, and is used interchangeably with, the phrase “including but not limited to” or “comprising”.

Any composition or method provided herein can be combined with one or more of any of the other compositions and methods provided herein.

### DETAILED DESCRIPTION OF THE EMBODIMENTS

Consumers and individuals who experience skin conditions and aging skin that adversely affect skin function and/or skin health, or detract from a robust or fit appearance, are interested in better methods of determining, diagnosing, characterizing, and identifying the causes of adverse, unhealthy, or unflattering skin conditions. Consumers and individuals are also concerned about more efficient provision and recommendation of directed treatment methods and therapeutic regimens by medical practitioners after aging skin or a skin condition has been determined, diagnosed, characterized, or identified to counteract more effectively and directly the problems of aging skin and/or various skin conditions or disorders that individuals experience.

Histological studies of the skin show that as aging occurs, the skin undergoes structural, functional, and metabolic changes that parallel the aging and degenerative changes in other body organs. While chronological and/or hormonal events play a significant role in the aging of skin, environmental stresses such as sun exposure may initiate and/or accelerate skin aging due to, in part, oxidative damage from overexposure to ultraviolet (UV) sunlight. In aged and/or aging skin, the cells may take longer to replenish, be less numerous, and/or breakdown more quickly. In particular, as aging occurs, the production of collagen is reduced while cell and tissue degradation is accelerated due to an overproduction of collagenase, i.e. a protease that breaks down collagen. The resulting dearth of collagen may lead to reduction in skin strength and elasticity, as well as skin that is fragile and more likely to shear.

Consumers and individuals are further interested in mitigating or delaying the dermatological signs of physiologically, chronologically or hormonally-aged or photo-aged skin, such as rhytids, fine lines, wrinkles, creases, drying, scaling, flaking and sagging, as well other skin conditions resulting from a progressive degradation of the skin matrix associated with a number of causes, for example, environmental assaults, physiological events, such as aging, or even psychological conditions, such as stress or tension.

The methods of the invention provide a more efficient and effective way to assess or diagnose skin aging, or a skin condition, such that the subject undergoing assessment or diagnosis receives a specific, customized, or personalized course of treatment based on the results of the methods without an extended time period following performance of the method.

### Age-Related Skin, Conditions and Disorders and Treatment

Skin disorders are frequently so common among older or aging individuals that it is often hard to distinguish normal changes from those related to a disorder. More than 90% of all older people have some type of skin disorder that may be associated with a more severe condition or disease. Thus, in embodiments, the present methods provide benefits for determining skin conditions and disorders in aging people or an aging population relative to normal changes in skin that are unrelated to an underlying condition or disorder. By way of nonlimiting example, skin conditions and disorders can be related to a variety of causes, including blood vessel diseases, e.g., arteriosclerosis, diabetes, heart/cardiovascular disease or congestive heart failure, liver disease, nutritional deficiencies, obesity, reactions to medicines and stress. Other causes of skin changes include, but are not limited to, allergies, e.g., plant allergies, food allergies and allergies to other substances; climate; clothing; exposure to industrial and household chemicals; and indoor heating. Sunlight (ultraviolet light) or photo-exposure, in particular, can result in a loss of skin elasticity (elastosis); noncancerous skin growths (keratoacanthomas); changes in pigment (liver spots); and thickening of the skin. Exposure to the sunlight has also been linked to skin cancers, including basal cell cancer, squamous cell carcinoma and melanoma. Skin changes may also be related to environmental factors, genetic makeup, nutrition, and other factors. In embodiments, the skin conditions are associated with changes in levels of one or more of the protein biomarkers set forth in Table 3 herein. In embodiments, the changes in levels of one or more of the protein biomarkers detected in a skin sample from a subject relative to control levels indicate an underlying condition that can be treated with a targeted therapy or therapeutic agent.

Skin also undergoes morphological and physiological changes concomitant with the aging of an individual. With aging, the epidermis (outer skin layer) thins, despite a stasis in the number of cell layers. The number of melanocytes (pigment-containing cells) decreases, while the remaining melanocytes increase in size. Aging skin often appears thinner, paler and more translucent than younger skin. Large pigmented spots, including liver spots, age spots, mottled skin, or lentigos, may appear in skin, particularly in areas exposed to the sun. Changes in the connective tissue reduce the skin's strength and elasticity, causing a condition known as elastosis, which is more noticeable in sun-exposed areas of the skin (solar elastosis). Elastosis produces a leathery, weather-beaten appearance, particularly of facial skin, that is common to farmers, sailors, and others who spend a large amount of time outdoors. As one ages, blood vessels of the dermis become more fragile, leading to bruising, bleeding under the skin (often called senile purpura), cherry angiomas, and similar conditions.

In addition, sebaceous glands produce less oil in an aging or older individual. While men usually experience a minimal decrease in the production of skin oils and usually after the age of 80, women gradually produce less oil beginning after menopause, thus, making it more difficult to maintain skin moisture, resulting in dryness and itchiness. With aging, the subcutaneous fat layer also thins so that it has less insulation and padding. This increases the risk of skin injury and reduces one's ability to maintain body temperature. Having less natural insulation directly relates to a susceptibility to hypothermia in cold weather. Because certain medications are absorbed by the fat layer, a loss or a decrease of this layer changes the mechanism of action of these types of medications. With aging, the sweat glands produce less sweat, making it harder to keep cool and increasing the risk for overheating or developing heat stroke. Growths, for example, skin tags, warts, rough patches (keratoses), discoloration, and other blemishes are more common in the skin of older and aging people.

The effects of skin changes with aging may be varied. As skin ages, an individual's risk for skin injury increases because skin is thinner, more fragile and contains less of its protective fat layer. A decrease in neurosensory function may also occur, characterized by an inability or a lesser ability to sense touch, pressure, vibration, heat, and cold. Rubbing or pulling on the skin can cause skin tears; fragile blood vessels can break easily; bruises, flat collections of blood (purpura); and raised collections of blood (hematomas) may form after even a minor injury. Pressure ulcers can be caused by skin changes, loss of the fat layer, reduced activity, poor nutrition, and illnesses. Sores are most easily seen on the outside surface of the forearms, but they can occur anywhere on the body. Aging skin undergoes repair more slowly than does younger skin. For example, wound healing may be up to four times slower in older or aged individuals, and this contributes to pressure ulcers and infections. Moreover, diabetes, blood vessel changes, lowered immunity, and other factors also affect the rate of skin healing and repair as an individual ages. The present methods are useful in determining the levels of skin biomarker proteins that are associated with the various conditions described supra so as to allow for improved efficacy in treating a subject whose skin assessment indicates aging skin and/or a given condition or disease, as well as to allow for the prevention of further detrimental or adverse effects to the skin of subject.

In various embodiments, the identification or determination of altered levels of one or more of the skin associated biomarkers of Table 3 relative to control levels may correlate with numerous types of skin conditions and disorders, for example, acne, rosacea, eczema, psoriasis, benign or malignant skin lesions, e.g., moles, freckles, discoloration, skin tags, lentigines, seborrheic keratoses, inflamed skin (rashes), dry skin, itchy skin, dermatitis, hives, vitiligo, epidermoid cysts, and the like. Skin conditions typical of aging include, without limitation, wrinkles (rhytids), fine lines, glabellar lines, lateral canthal lines (crow's feet), dry skin, loose or sagging skin, hyperpigmentation, hypopigmentation, and the like, particularly, but certainly not exclusively, on the face. Following the identification or determination of the biomarker levels associated with a skin condition or disorder in a subject who has undergone assessment, a directed treatment can be administered to the subject. Furthermore, the progress of a treatment or therapy may be monitored or followed by periodic testing of the subject's skin sample for a change in the levels of the one or more biomarkers, particularly a change toward normal, healthy levels based on those in normal, healthy controls. In an embodiment, the biomarkers detected and monitored include one or more, or a subset, of those in Table 3.

### Methods of Assessment of Skin Biomarkers

The present invention provides methods for assessing skin conditions, e.g., disease or injury, and/or for assessing aging skin or degree or extent of aging in skin. In an embodiment, the methods involve the use of quantitative proteomics analysis. The degree of aging in skin refers to the extent or intensity of aging signs that are exhibited in and on the skin, e.g., fine lines, wrinkles, creases and sagging, due to a subject's chronological age. It will be appreciated that skin wrinkles, creases, lines, pigmentation aberrations or alterations, weakness and sagging progress with age. Moreover, in addition to age, environmental factors such as exposure to ultraviolet rays can exacerbate skin lines, flaccidness, and dullness of complexion and pigmentation. Skin lines and flaccidness can be measured using known methods and scale products (e.g., US20130079643 A1; H. Ohshima et al., 2011, Skin Res. Technol., 17(1): 101-107; Y. Harth et al., 2011, J. Cosmetic Dermatol., 10(1):24-29; Merz Aesthetics Scales™ (Merz Pharmaceuticals, Frankfurt, Germany; M. A. Kane et al., 2012, Aesthetic Surgery Journal, 32(3):275-285).

It will be further appreciated that when the skin beings to sag or to form lines and wrinkles with age, the ability of epidermal cells, skin fibroblasts and other skin cells to proliferate attenuates in the skin. Thus, a reduction or decrease in the proliferation ability of skin cells can serve as an indicator of the degree of skin aging and senescence. Of interest, senescence-associated β-galactosidase (SA-β-Gal), which is active at pH 6, has been reported to increase in epidermal keratinocytes and dermal fibroblasts that have been aged through repeated passages in culture; the increase in SA-β-Gal is correlated with aging of human skin (G. Dimri et al., 1995, Proc. Natl. Acad. Sci. USA, 92:9363-9367). This lends support to the finding that proteins identified in aging skin samples, which comprise epidermal keratinocytes and fibroblasts, can serve as biomarkers for human skin aging.

The skin samples for use in performing the present methods, and the methods for obtaining skin samples from a subject in need, test and control subjects, are not intended to be limiting. As noted herein, a number of procedures can be used for obtaining a skin sample from a subject, for example, scraping, e.g., mechanical scraping, swabbing and/or direct elution, pressure blotting, electroblotting (electric transfer), and the like, can be used. In embodiments, a skin sample is obtained by the use of a ‘tape strip’ or a ‘tape disk,’ which has adhesive properties and is applied to the subject's skin in a prescribed manner and the material is removed for the purposes of subjecting the skin sample to protein/proteomic analysis.

In accordance with the present invention, skin samples encompass skin tissue and skin cells. Skin tissue and cells may be obtained from a variety of species, including, for example, humans, non-human primates, pigs, dogs, rats, mice, rabbits, and other mammals. In preferred embodiments, human skin or human-derived tissues is used, particularly to determine and treat skin aging. Skin biopsy samples, cultured skin tissue, and cultured skin cells, and the like, obtained from skin biopsy samples can be used to determine the degree or extent of skin aging. By way of example, skin cells may include keratinocytes (epidermal keratinocytes), skin fibroblasts, melanocytes, Merkel cells, Langerhans cells, mast cells, endothelial cells, sebum cells, follicular cells, hair papilla cells and hair matrix cells, etc. Skin cells may be obtained, e.g., for control purposes, from the American Type Culture Collection (ATCC), Manassas, Va., or are commercially available from Takara Bio/Clontech (Mountain View, Calif.) or PromoCell, Heidelberg, Germany). Skin tissue examples include the epidermis, the dermis and the horny cell layer, etc., and can be purchased from BioChain Institute, Inc. (Newark, Calif.) or SuperBioChips Laboratories, Seoul, Korea).

Skin biopsy samples may be skin cells, tissues, or cells derived from skin tissue. In a particular embodiment, skin biopsy samples are from the outermost layer of the epidermis, the stratum corneum (‘horny layer’) and may be obtained, for example, by tape stripping as described herein. Skin cells may also be collected, for example, by the use of a ‘horny cell layer checker,’ which measures the degree of parakeratosis and cell area of the horny cell layer and has been used to evaluate the degree or extent of skin roughness and turnover rate of the horny cell layer, as mentioned in U.S. Pat. No. 7,972,788.

The methods of the invention allow for the diagnosis or determination of skin conditions or factors involved in aged versus young (non-aged) skin by assessing changes or alterations, a decrease or increase, in the levels of one or more skin-associated protein biomarkers in skin samples obtained from a subject undergoing assessment compared with a suitable control. The methods of the invention also allow for the diagnosis or determination of skin conditions or factors involved in aged versus young (non-aged) skin by assessing changes or alterations, e.g., a decrease or increase, in the levels of a subset of skin-associated protein biomarkers in skin samples obtained from a subject undergoing assessment compared with a suitable control. In an embodiment, the one or more protein biomarkers are as set forth in Table 3 herein. In an embodiment, the one or more protein biomarkers are a subset or panel of the proteins presented in Table 3 herein. In an embodiment, the subset or panel of protein biomarkers encompasses one or more heat shock proteins (HSP), including members of the HSP70 family, such as one or more of HSPA9, HSPA5 and HSPA8. In a particular embodiment, the subset of HSPs includes HSPA9, HSPA5 and HSPA8. In an embodiment, the subject undergoing assessment exhibits a skin condition or disease and the control does not. In an embodiment, the subject undergoing assessment has aged or aging skin and the control is a younger subject, e.g., of a young age group, who does not have aged or aging skin. In embodiments, the control is a healthy individual without aging skin or signs thereof and/or without disease, skin disease, or injury. In an embodiment, the change in the levels of the one or more skin-associated protein biomarkers in the control (e.g., young, non-aged skin) versus levels of the protein biomarkers in a sample from a subject undergoing testing is a fold-increase, such as shown in Table 3 herein, for example, an increase of about or equal to 1.5 fold or greater, such as 1.6 fold, 1.7 fold, 1.72 fold, 1.73 fold, 1.74 fold, 1.75 fold, 1.76 fold, 1.77 fold, 1.78 fold, 1.79 fold, 1.8 fold, 1.9 fold, 2 fold, 3 fold, 4 fold, 4.1 fold, 4.2 fold, 4.3 fold, 4.4 fold, 4.5 fold, 4.6 fold, 4.7 fold, 4.8 fold, 4.9 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, or 20 fold, or greater, as well as values and sub-values between the listed values. In an embodiment, the change in the levels of the one or more skin-associated protein biomarkers in the control (e.g., young, non-aged skin) versus levels of the protein biomarkers in a sample from a subject undergoing testing is a fold-decrease, such as shown in Table 3 herein, for example, a decrease of about or equal to 1.2 fold, 1.21 fold, 1.23 fold, 1.24 fold, 1.25 fold, 1.26 fold, 1.27 fold, 1.28 fold, 1.29 fold, 1.3 fold, 1.31 fold, 1.32 fold, 1.33 fold, 1.34 fold, 1.35 fold, 1.36 fold, 1.37 fold, 1.38 fold, 1.39 fold, 1.4 fold, 1.41 fold, 1.42 fold, 1.43 fold, 1.44 fold, 1.45 fold, 1.46 fold, 1.47 fold, 1.48 fold, 1.49 fold, 1.5 fold, 1.51 fold, 1.52 fold, 1.53 fold, 1.54 fold, 1.55 fold, 1.56 fold, 1.57 fold, 1.58 fold, 1.59 fold, 1.6 fold, 1.61 fold, 1.62 fold, 1.63 fold, 1.64 fold, 1.65 fold, 1.66 fold, 1.67 fold, 1.68 fold, 1.69 fold, 1.7 fold, 1.71 fold, 1.72 fold, 1.73 fold, 1.74 fold, 1.75 fold, 1.76 fold, 1.77 fold, 1.78 fold, 1.79 fold, 1.8 fold, 1.81 fold, 1.82 fold, 1.83 fold, 1.84 fold, 1.85 fold, 1.86 fold, 1.87 fold, 1.88 fold, 1.89 fold, 1.9 fold, 1.95 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.57 fold, 3.6 fold, 3.67 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, 12 fold, or 12.5 fold, as well as values and sub-values between the listed values.

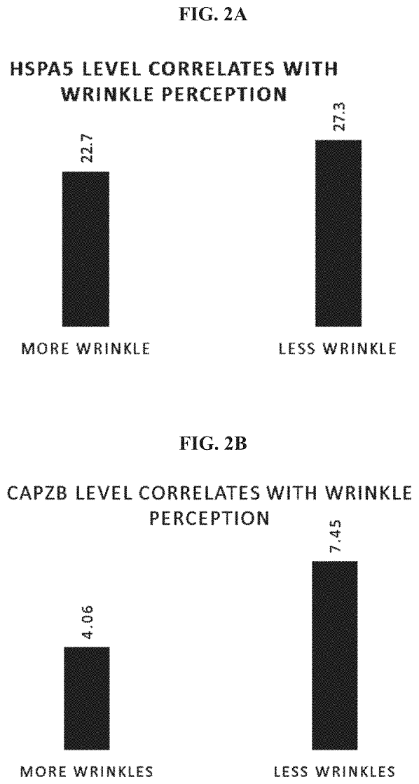

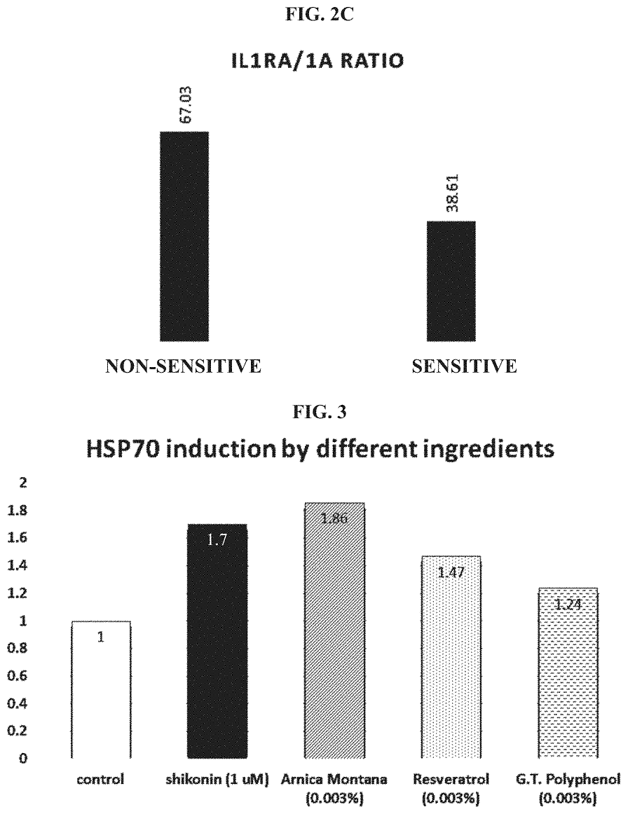

As will be appreciated by the skilled practitioner, HSP70 proteins are chaperone proteins that play an essential role in cell homeostasis by aiding in the folding of cellular proteins into their correct three-dimensional structures for optimum protein function. In addition, HSP70 proteins can correct misfolded proteins after various stresses, such as ultraviolet (UV) light exposure. The level of HSP70 decreases with age in skin and correlates with wrinkle perception (see, e.g., FIG. 2A). As discussed infra, boosting HSP70 levels, such as by providing one or more agents or compounds that induce HSP70 levels in skin as shown in FIG. 3, may reduce UV-induced wrinkles in a subject in need. Such HSP70-inducing agents or compounds, e.g., shikonin, Arnica montana, resveratrol, green tea derived epigallocatechin-3-gallate (EGCG), may provide advantageous anti-aging skin benefits for subjects in need.

In embodiments, the methods of the invention are useful for a variety of assessments of skin conditions and for assessing the effects of aging. In one aspect, severity of a skin condition can be evaluated based on a change, either an increase or decrease, in the level of or fold-expression of one or more specific biomarkers associated with a specific skin condition, disease, insult, or injury or with the aging of skin. In certain instances, severity of the condition or aging can also be assessed by identifying a change in a biomarker expression pattern, or subset of biomarkers, known to follow a specific disease or aging condition. For example, a specific condition or aging status of skin may give rise to a specific biomarker expression pattern that is replaced, in one or more phases, with one or more specific expression patterns as the resulting condition deteriorates or improves.

In an embodiment, a specific condition or aging status of skin correlates with a change, e.g., an increase or decrease, in levels of a subset of the skin protein biomarkers set forth in Table 3 herein compared with levels in an appropriate control. In a particular embodiment, a decrease in the levels of a subset of the protein biomarkers of Table 3 in a subject's skin versus control levels is associated or correlated with skin aging or with a skin attribute, e.g., an adverse skin attribute, such as wrinkling, creasing, folding, and abnormal pigmentation, such as hyper- or hypo-pigmentation. In an embodiment, the subject whose assayed skin sample shows a decrease in the levels of one or more of the protein biomarkers of Table 3 is treated, or is recommended for treatment, with an agent that targets the protein biomarkers so as to increase their levels relative to control, so as to obtain levels of these proteins similar to control levels. By way of nonlimiting example, a p value of <0.05 (p<0.05) reflects a statistically significant change (e.g., increase or decrease) of a protein biomarker level relative to control reflects. In an embodiment, the one or more protein biomarkers whose levels are found to decrease and correlate with aging and/or an adverse skin attribute include one or more of the heat shock proteins, e.g., HSPA9, HSPA5 and HSPA8.

In yet another embodiment, an improvement in skin condition or in aging skin can be assessed in which a specific biomarker expression pattern, or subset of biomarkers, associated with a specific skin condition or with aging skin is first identified and subsequently observed to be more similar to the biomarker expression pattern observed to be associated with control, or normal skin. Such an assessment may be observed to follow a change in biomarker presentation following an improvement or change in the condition or disease, or may follow therapeutic or prophylactic intervention or therapy. Accordingly, assessment of the efficacy of particular skin treatments for aging or disease is an aspect of the present methods. In certain cases, assessment of the efficacy of treatment or the state of health of skin involves comparing samples obtained from the same subject and taken at different or varying time points.

### Correlation Between Biomarkers and Skin Attributes

In accordance with an aspect of the present invention, the faces of subjects undergoing skin testing were graded by a dermatologist for the following visible or tactile attributes: fine wrinkles, coarse wrinkles, mottled pigmentation, discreet pigmentation, erythema and blotchiness, using a scale from 0-10, with 10 as the most pronounced, prominent, or noticeable skin attribute and 0 as the least pronounced, prominent, or noticeable skin attribute. In an embodiment, five subjects within a narrow age range (i.e., ages 69, 69, 71, 71 and 73) were selected for the correlation study in order to exclude age as a factor affecting or influencing the result. The correlation between biomarkers and skin attributes, especially adverse or unflattering skin attributes, was calculated using Pearson-correlation coefficient. For protein biomarkers identified from the proteomic study as described in Example 1, heat shock proteins showed good correlation with various skin attributes, particularly those skin attributes that are considered to be unpleasant, undesirable, or unwanted by the subject. For example, it was found that a high correlation existed between the level of the HSPA5 protein and the presence of coarse wrinkles (r²=0.95) and mottled pigmentation (r²=0.89) in the skin.

In another aspect, the correlation between protein biomarkers and skin attributes was assessed in a study in which the subjects undergoing testing were queried regarding their personal opinions about their own skin attributes prior to the assessment study, as described in Example 2 and shown in FIGS. 2A-2C.

### Methods and Assays for Skin Biomarker Detection

### Immunoassay

In embodiments, the biomarkers, such as skin-associated protein biomarkers, of the present invention can be detected, assessed and/or measured by an immunoassay. Immunoassays involve the use of capture reagents, such as antibodies, that specifically bind to (“capture”) the biomarkers. Antibodies can be produced by methods well known in the art, e.g., by immunizing animals with the biomarkers, or by recombinant methods using molecular biology techniques routinely practiced in the art. Many antibodies are commercially available. Using immunoassays, biomarkers can be identified in and/or isolated from samples based on their binding characteristics. Immunoassays such as ‘sandwich’ immunoassays including ELISA or fluorescence-based immunoassays, immunoblots, such as Western Blots, and other enzyme immunoassays are suitable for use according to the present invention. Other assays include nephelometry, which is performed in liquid phase, in which antibodies are in solution. When an antibody binds to an antigen, a change in absorbance results, and this change can be measured. In a SELDI (Surface-Enhanced Laser Desorption/Ionization)-based immunoassay, a specific capture reagent for the biomarker is attached to the surface of a mass spectrometry (MS) probe, e.g., a pre-activated protein chip array (biochip) for covalently coupling proteins or peptides. The biomarker is then specifically captured on the biochip through this reagent, and the captured biomarker is detected by mass spectrometry. The ProteinChip SELDI system (Bio-Rad, Hercules, Calif.) combines surface-enhanced selective capture with high-sensitivity mass spectrometry for protein biomarker profiling and immunoassay applications

Although antibodies are useful because of their extensive characterization, any other suitable agent (e.g., a peptide, an aptamer, or a small organic molecule) that specifically binds a biomarker, for example, a protein biomarker associated with or extracted from skin, may be employed in lieu of an antibody in the above described immunoassays. For example, an aptamer, which is a nucleic acid-based molecule that specifically binds a specific ligand, e.g., a skin-associated biomarker, could be used. Methods for making aptamers with a particular binding specificity are known in the art and are described, for example, in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

### Electrochemiluminescent Assay

In embodiments, the skin associated protein biomarkers determined by the methods of the present invention may be detected by a electrochemiluminescent detection system, which involves the use of labels that emit light when electrochemically stimulated (e.g., Meso Scale Discovery (Rockville, Md.). In such a system, background signals are minimal because the electrical stimulation mechanism is de-coupled from the signal (light). In addition, the labels are stable and non-radioactive and utilize convenient coupling chemistries. The labels also emit light at 620 nm, thus avoiding problems with color quenching. Such systems are described, for example, in U.S. Pat. Nos. 7,497,99; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434, as well as U.S. Patent Application Publication Nos. 2009/0170121; 2009/006339; 2009/0065357; 2006/0172340; 2006/0019319; 2005/0142033; 2005/0052646; 2004/0022677; 2003/0124572; 2003/0113713; 2003/0003460; 2002/0137234; 2002/0086335; and 2001/0021534.

### Other Biomarker Detection Methods

The skin associated biomarkers, e.g., protein biomarkers, can also be detected by other methods as frequently used in the art. For example, such detection systems include optical methods, electrochemical methods (voltametry and amperometry techniques), atomic force microscopy, and radio frequency methods, e.g., multipolar resonance spectroscopy. By way of example, optical methods include confocal and non-confocal microscopy, as well as the detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

As mentioned above, a sample, such as a skin sample, may also be analyzed by means of a biochip. In general, biochips are solid substrates that have a planar surface to which a capture reagent (also called an adsorbent or affinity reagent) is attached. The surface of a biochip generally contains a plurality of addressable locations onto which the capture reagent is bound. Protein biochips are biochips adapted for the capture of polypeptides, proteins, and peptides. Many protein biochips are available and are known to those having skill in the art. For example, protein biochips include those produced by Affymetrix, Inc. (Fremont, Calif.), Zyomyx (Hayward, Calif.), Invitrogen Corp. (Carlsbad, Calif.), Ciphergen Biosystems, Inc. (Fremont, Calif.), R&D Systems, Inc. (Minneapolis, Minn.), Biacore (Uppsala, Sweden) and Procognia (Berkshire, UK). Protein biochips are described, for example, in U.S. Pat. Nos. 6,537,749; 6,329,209; 6,225,047; and 5,242,828; and in International PCT Publication Nos. WO 2000/56934; and WO 2003/048768.

### Proteomics Assays

A variety of assays are commonly used in proteomics studies to assess protein biomarkers and alterations in the levels of such proteins, particularly compared with control levels. By way of nonlimiting example, such assays include Western Blot analysis or immunohistochemistry with labeled antibodies; mass spectrometry, flow cytometry, microfluidics, microscopy, deep sequencing and protein microarray. (See, e.g., M. Breker and M. Schuldiner, 2014, Nature Reviews Mol. Cell Biol., 15:453-464). In accordance with the present methods, the protein biomarkers are skin-associated proteins and peptides that show changes in levels, or amounts, for example, depending on the age and the physical health of a subject, and more specifically, depending on the aging status and/or physical condition of the subject's skin. In a particular embodiment, the skin-associated protein biomarkers set forth in Table 3 herein showed differences in levels in older subjects relative to younger subjects whose skin samples were assayed and compared. See, e.g., Example 1.

### Biomarker Panels

The skin-associated protein biomarkers of the present invention can be used in diagnostic tests to assess, determine, identify and/or qualify (used interchangeably herein) aging status of a subject and/or an underlying condition, damage, injury, or disease in a subject. The phrases “skin aging status” or “skin condition status” include any distinguishable manifestation of the aging and/or condition of a subject's skin, including, without limitation, wrinkles, creases, fine lines, loss of elasticity, loss of pliancy, loss of firmness, and the like, as well as not having aging skin, i.e., having youthful, non-aged, firm, uninjured and supple skin. For example, skin aging or skin condition status includes, without limitation, the presence or absence of aged, damaged, injured, or diseased skin in a subject; the risk of a subject's developing aged, damaged, injured, or diseased skin; the stage or severity of a subject's aged, damaged, injured, or diseased skin; the progress (e.g., over time) of skin aging, damage, injury, or disease; and the effectiveness or response to treatment of aging, damaged, injured, or diseased skin (e.g., clinical follow up and surveillance of the skin after treatment). Based on this status, further procedures may be indicated for a subject, including additional diagnostic tests or therapeutic procedures, treatments, or regimens.

The power of a diagnostic test to correctly predict a status is typically measured as the sensitivity of the assay, the specificity of the assay or the area under a receiver operated characteristic (“ROC”) curve. Sensitivity refers to the percentage of true positives that are predicted by a test to be positive, while specificity refers to the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value refers to the percentage of individuals who test positive who are actually positive. Negative predictive value refers to the percentage of individuals who test negative who are actually negative. In particular embodiments, the biomarker panels or subsets of biomarkers detected by the present methods may show a statistical difference in different skin aging or skin condition statuses of at least p<0.05, p<10⁻², p<10⁻³, p<10⁻⁴, or p<10⁻⁵. Diagnostic tests that use these biomarkers may show an ROC of at least about 0.5, at least about 0.6, at least about 0.7, at least about 0.8, or at least about 0.9.

In an embodiment, the skin associated biomarkers are differentially present or differentially expressed in aging and/or diseased skin versus non-aged and/or healthy skin and, therefore, are useful in aiding in the determination of aging and health status. In certain embodiments, the biomarkers are measured in a subject's sample using the methods described herein and compared, for example, to predefined protein biomarker levels and correlated to skin aging or disease status. In particular embodiments, the measurement(s) may then be compared with a relevant diagnostic amount(s), cut-off(s), or multivariate model scores that distinguish a positive skin aging or disease status from a skin aging or disease status. The diagnostic amount(s), etc. represents a measured amount of a biomarker(s) above which or below which a subject is classified as having a particular skin aging or disease status. For example, if the skin-associated biomarker(s) associated with aging or disease is/are up-regulated or over expressed compared to a control, then a measured amount(s) above (greater than) the diagnostic cutoff(s) provides a determination or diagnosis of skin aging or disease. Alternatively, if the skin-associated biomarker(s) associated with aging or disease is/are down-regulated or under expressed compared to a control, then a measured amount(s) at or below the diagnostic cutoff(s) provides a determination or diagnosis of skin aging or disease. As will be appreciated by those having skill in the art, by adjusting the particular diagnostic cut-off(s) used in an assay, the sensitivity or specificity of the diagnostic assay may be increased depending on the preference of the diagnostician. In certain embodiments, the particular diagnostic cut-off can be determined, for example, by measuring the amount of biomarkers in a statistically significant number of samples from subjects with the different skin aging and/or disease statuses, and establishing the cut-off to suit the desired levels of specificity and sensitivity. As the skilled practitioner will also appreciate, there are many ways to use the measurements of two or more biomarkers in order to improve the diagnostic question under investigation. In a straightforward, but often effective approach, a positive result is assumed if a sample is positive for at least one of the biomarkers investigated.

In certain other embodiments, the values measured for the protein members of a biomarker panel are mathematically combined and the combined value is correlated to the underlying diagnostic question. Biomarker values may be combined by any appropriate mathematical method practiced by those having skill in the art. Well-known mathematical methods for correlating a marker combination to an aging or disease status employ procedures and parameters such as discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The person of skill in the art will readily be able to select an appropriate method to evaluate a biomarker combination in accordance with the present invention. In one embodiment, the method used in a correlating a biomarker combination of the present invention, e.g. to determine or diagnose aging or injury or disease, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. These statistical methods are described, for example, in Ruczinski et al., 2003, J. Computational and Graphical Statistics, Vol. 12, pp. 475-511; Friedman, J. H., 1989, J. American Statistical Association, Vol. 84, pp. 165-75; Hastie, Trevor, Tibshirani, Robert, Friedman, Jerome. The Elements of Statistical Learning, Springer Series in Statistics (2001); Breiman, L., Friedman, J. H., Olshen, R. A., Stone, C. J. Classification and regression trees, California: Wadsworth (1984); Breiman, L., 2001, Machine Learning Vol. 45, pp. 5-32; Pepe, M. S., The Statistical Evaluation of Medical Tests for Classification and Prediction, Oxford. Statistical Science Series, Vol. 28 (2003); and Duda, R. O., Hart, P. E., Stork, D. G., Pattern Classification, Wiley Interscience, 2nd Edition (2001).

### Determining Therapeutic Efficacy of a Drug or Treatment

In an embodiment, the present invention provides methods for determining the therapeutic efficacy of a pharmaceutical drug or treatment to reduce, eliminate, ameliorate, abrogate, diminish, or cure a skin condition, injury or aging. The methods are useful for performing clinical studies of a drug or pharmaceutical, as well as monitoring the progress of a subject undergoing treatment with the drug or pharmaceutical. Therapy or clinical trials involve administering the drug or pharmaceutical in a particular regimen, which may involve a single dose or multiple doses of the drug or pharmaceutical over a predetermined period of time. The medical practitioner or clinical researcher monitors the effect of the drug on the subject over the course of administration. If the drug has a pharmacological impact on the condition, the amounts or relative amounts (e.g., the pattern or profile) of one or more of the protein biomarkers of the invention may change or be altered toward a non-aging or non-disease profile. The course of one or more biomarkers can be followed or monitored in the subject during the course of treatment or therapy. Thus, in an aspect, the method of the invention involves measuring one or more biomarkers in a subject receiving treatment or therapy with a drug or pharmaceutical, and the like, and correlating the biomarker levels with the skin aging and/or disease status of the subject (e.g., by comparison to predefined levels of the biomarkers that correspond to different skin aging and/or diseases statuses, including no skin aging or disease). An embodiment of the method involves determining the levels of one or more biomarkers at at least two different time points during a course of drug treatment or therapy, e.g., at a first time and at a later second time, and comparing the change in levels of the biomarkers, if any. By way of example, the levels of one or more biomarkers in Table 3, e.g. the HSP biomarkers, such as HSPA9, HSPA5 and HSPA8 members of the HSP70 family, can be measured before and after drug administration or at two different time points during drug administration. The effect of therapy is determined based on the comparisons. If a treatment is effective, then the one or more biomarkers will trend toward normal (non-aged, non-diseased or non-injured skin comparators) expression values. By contrast, if treatment is ineffective, the one or more biomarkers will trend toward expression values of aged, diseased, or injured skin comparators.

In accordance with the invention, certain compounds or agents (pharmaceutical agents) can induce levels of HSP70 proteins in skin. By way of illustrative and nonliming example, the following were identified as agents/compounds that boosted (induced or increased) HSP70 protein levels relative to control level in skin (FIG. 3): shikonin (C₁₆H₁₆O₅; a naturally occurring naphthoquinone compound, which is the main component of red pigment extracts from Lithospermiun erythrorhizon Sieb et Zucc of East Asia), Arnica montana (referred to as “wolf's bane” is an ethnobotanical European flowering plant in the sunflower family. The primary constituents of Arnica montana include essential oils; fatty acids, pseudoguanianolide sesquiterpene lactones and flavanone glycosides. Helenalin is a pseudoguanianolide sesquiterpene toxin derived from the head of the Arnica montana plant and is not harmful in small quantities.), resveratrol (a trans-3,5,4′-trihydroxystilbene (C₁₄H₁₂O₃), stilbenoid or natural phenol produced in several plants in response to injury by pathogens. Food sources of this compound include grapes, raspberries, blueberries and mulberries); and G. T. polyphenol (i.e., the green tea catechin, epigallocatechin-3-gallate (EGCG)). In an embodiment, HSP70 level correlates inversely with the presence of wrinkles in a subject, particularly an aging or aged subject. Thus, the administration of one or more of the above products, as well as other products that boost HSP70 level, to a subject whose skin sample is found to indicate low level of HSP70 (and who has or is perceived as having an aging skin phenotype or condition, such as wrinkles) may be provided to reduce the aging skin phenotype or condition, such as wrinkles, in the subject in accordance with the methods described herein.

Pharmaceutical compositions are also provided comprising an effective amount of an HSP70 protein inducing agent and one or more pharmaceutically acceptable excipients, carriers, or diluents. With the methods described herein, several inducing agents have been identified to increase HSP70 protein levels when administered at an amount capable of inducing production of HSP70 proteins in cells. In certain embodiments, the pharmaceutical composition comprising an HSP70 protein inducing agent may be administered following identification of the skin condition or disorder in a subject by measurement of the levels of one or more skin biomarker proteins in a skin sample from a subject. For example, extracts of Tiliacora triandra have been shown to result in a statistically significant increase in HSP70 protein level when administered at greater than 0.01% by weight of the composition (e.g., more than 0.05%, more than 0.075%, etc.). Pharmaceutical compositions comprising extracts of Tiliacora triandra may be administered to a subject identified as having decreased level of HSP70 proteins in their skin. Methods of preparing and administration of Tiliacora triandra extracts are provided in U.S. Pat. Nos. 8,771,758, 9,238,000, and 10,076,479, each hereby incorporated by reference in their entirety and specifically in relation to Tiliacora triandra extracts. Similarly, extracts of Portulaca oleracera have been shown to result in a statistically significant increase in HSP70 protein level when administered at greater than 0.01% by weight of the composition (e.g., more than 0.05%, more than 0.075%, etc.). Methods of preparing and administration of Portulaca oleracera extracts are provided in U.S. Pat. No. 9,149,665, hereby incorporated by reference in its entirety and specifically in relation to Portulaca oleracera extracts. In certain embodiments, the HSP70 protein inducing agent may be an extract of Agania spinosa (e.g., argan oil) as described in U.S. Pat. No. 8,178,106, hereby incorporated by reference in its entirety and specifically in relation to Argania spinosa extracts. In various implementations, the extract may be an aqueous or nonaqueous extract. In certain embodiments, the extract may be prepared by an extraction medium that is water, lower alkyl (e.g., methanol, ethanol, etc.), or combinations thereof.

In various implementations the pharmaceutical composition may comprise a therapeutically effective amount of a HSP70 protein inducing agent such as Tiliacora triandra extract (e.g., more than 0.01% by weight of the composition or more than 0.02% by weight of the composition or more than 0.05% by weight of the composition or more than 0.075% by weight of the composition or between 0.02% and 0.5% by weight of the composition etc.), Portulaca oleracera extract (e.g., more than 0.01% by weight of the composition or more than 0.02% by weight of the composition or more than 0.05% by weight of the composition or more than 0.075% by weight of the composition or between 0.02% and 0.5% by weight of the composition etc.), cannabidiol (e.g., more than 0.001% by weight of the composition or more than 0.002% by weight of the composition or more than 0.005% by weight of the composition or more than 0.0075% by weight of the composition or between 0.002% and 0.1% by weight of the composition etc.), Arnica montana extract (e.g., more than 0.001% by weight of the composition or more than 0.002% by weight of the composition or more than 0.005% by weight of the composition or more than 0.0075% by weight of the composition or between 0.002% and 0.5% by weight of the composition etc.), shikonin (e.g., more than 0.1 μM or more than 0.2 μM or more than 0.5 μM or between 0.1 μM and 10 μM etc.), reservatrol (e.g., more than 0.001% by weight of the composition or more than 0.002% by weight of the composition or more than 0.005% by weight of the composition or more than 0.0075% by weight of the composition or between 0.002% and 0.5% by weight of the composition etc.), green tea polyphenol (e.g., more than 0.001% by weight of the composition or more than 0.002% by weight of the composition or more than 0.005% by weight of the composition or more than 0.0075% by weight of the composition or between 0.002% and 0.5% by weight of the composition etc.), argan oil (e.g., more than 0.1% by weight of the composition or more than 0.2% by weight of the composition or more than 0.5% by weight of the composition or more than 0.75% by weight of the composition or between 0.2% and 5% by weight of the composition, or between 0.2% and 2.5% by weight of the composition, etc.), butyric acid (e.g., more than 0.1% by weight of the composition or more than 0.2% by weight of the composition or more than 0.5% by weight of the composition or more than 0.75% by weight of the composition or between 0.2% and 5% by weight of the composition, or between 0.2% and 2.5% by weight of the composition, etc.), and combinations thereof.

Any of a number of procedures for obtaining a skin sample from a subject can be employed to practice the methods described herein. In some embodiments, scraping, e.g., mechanical scraping, swabbing and/or direct elution, pressure blotting, electroblotting (electric transfer), and the like, can be used. In an embodiment, a skin sample is obtained by the use of a ‘tape strip’ or a ‘tape disk.’ For such use, a material of known adhesive properties (“tape”) is applied to the subject's skin in a prescribed manner and the material is removed for the purposes of subjecting the skin sample to proteomic analysis or evaluation. Adhesive material that is suitable for obtaining skin samples via ‘tape-stripping,’ are not required to be in a specific ‘strip’ shape or format, but can be of any shape or format, such as a disk tape or adhesive. Such adhesive or tape materials include, without limitation, adhesive tapes such as D-SQUAME™ and SEBUTAPE™ (CuDerm Corporation, Dallas, Tex.) or BLENDERM™ and SCOTCHTAPE™ (3M Company, St. Paul, Minn.), and hydrogels such as HYPAN™ (Hymedix International, Inc., Dayton, N.J.), and other types of materials with adhesive properties or appropriate ‘stickiness,’ such as glues, gums, and resins.

Regardless of the method used for obtaining a skin sample to assess the skin associated biomarkers for analysis, in most instances, the biomarkers, such as protein biomarkers or nucleic acid biomarkers, can be removed or extracted from the device or liquid used to obtain the sample, if desired, and processed in such a manner or by an analytical method that allows for assessment of the biomarkers, e.g. via mass spectrometry (MS), capillary electrophoresis (CE), liquid chromatography (LC), nuclear magnetic resonance spectroscopy (NMR), and the like, as known to those having skill in the art. In general, the chosen method of analysis will determine how a sample is processed using sample processing techniques that are commonly used by those having skill in the art. In a particular embodiment, the skin associated biomarkers for analysis are protein biomarkers, such as those set forth in Table 3, or a subset or panel thereof, which may include, illustratively, one or more of HSPA9, HSPA5, HSPA8.

### Other Methods of the Invention

The present methods provide the identification of substances (compounds, small molecules, agents, or drugs), i.e., candidate substances, that are effective in treating or preventing the aging of skin, in which a substance can modulate, e.g. decrease or increase, the levels of one or more proteins of Table 3, or protein biomarker profiles correlated with aged or aging skin. In an embodiment, the method identifies a substance that decreases the levels of one or more proteins of Table 3. In an embodiment, the method identifies a substance that increases the levels of one or more proteins of Table 3. In an embodiment, the method involves contacting a skin sample with a candidate substance under suitable conditions for a predetermined period of time. In an embodiment, the skin sample comprises skin cells or tissue, and the candidate substance is added to a culture of the skin cells or tissue, in which the substance contacts the cells or tissue for a specified time. In an embodiment, the candidate substance is directly applied to the skin sample. Following contact and/or culture of the skin sample and the candidate substance, the levels of one or more of the proteins of Table 3 are determined, e.g., by quantification via an appropriate or suitable method, wherein the proteins change their levels with the aging of skin. Thereafter, the effect of the candidate substance on the protein levels of the skin sample undergoing testing is evaluated by comparing the levels of the proteins of the test skin samples with the levels of the proteins in a control skin sample, or skin cell or tissue sample. In an embodiment, the control skin sample is the same as that of the test skin sample, except that the control sample is not contacted with any candidate substance. In embodiments, the candidate substance (compound, small molecule, agent, or drug), may be a protein, peptide, vitamin, hormone, polysaccharide, oligosaccharide, monosaccharide, low-molecular weight organic compound, synthetic compound, nucleic acid (DNA, RNA, oligonucleotide, mononucleotide, etc.), lipid, other natural compound, or any combination thereof.

As noted above, the way in which a candidate substance is made to contact a skin sample (or skin cells or tissue) is not intended to be limiting. For example, the candidate substance may be applied directly onto or over the skin sample, or the candidate substance may be administered to a test animal, for example a mammal such as a human, pig, dog, rabbit, guinea pig, rat, or mouse, via a suitable route, including oral, intravenous, subcutaneous, peritoneal, orbital, and the like. In addition, skin cell and/or tissue samples may be cultured in a culture container or on a substrate onto which the candidate substance has been applied, coated, impregnated, or affixed.

The time period during which the skin sample, e.g., skin cells and/or tissue, remains in contact or culture with a candidate substance is also not intended to be limiting. Any desired time period may be established, provided that it is long enough to determine whether the candidate substance has any effect on the levels of one or more of the proteins of Table 3, or their encoding genes, in a skin sample (e.g., in skin cells and/or skin tissues). By way of example, if normal human epidermal keratinocytes are used as skin cells, a culture time of 12 to 48 hours or 12 to 24 hours is suitable. As referred to herein, cultured skin cells and/or skin tissue grow and proliferate during their time in culture. Control skin samples, e.g., skin cells and/or skin tissues, which are used as comparators with a test skin sample (skin cells or tissue) are those that have not been contacted by the candidate substance. Frequently, the control skin samples, e.g., skin cells and/or skin tissues, are subjected to the same treatment conditions as the test skin sample, except that the controls are not contacted with the candidate substance.

By way of particular example, the levels or amount of expression of the following subset of proteins from Table 3 (Example 1) herein: GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD was found to decrease in the skin of aged or old subjects versus young controls by the practice of the described methods. Accordingly, an optimal treatment or therapeutic product or regimen for aging skin would be that which caused an increase in the levels of such proteins in the skin of aged subjects, for example, toward levels in the skin of young, non-aged control subjects. Thus, if a treatment or therapeutic product or regimen involving contact of the skin, skin tissue, or skin cells of an aged or old subject is evaluated and found to increase the levels or amount of expression of any of these biomarker proteins relative to the control, then the treatment or therapeutic product or regimen may be identified as being effective in treating or preventing the aging of skin. In a particular embodiment, the one or more proteins of Table 3 whose levels change, i.e., increase, in the skin of young versus old subjects is a subset of proteins that includes heat shock proteins HSPA9, HSPA5 and HSPA8. Accordingly, a directed treatment or therapy for an aged subject whose skin is identified as having a deficiency in one or more of these proteins would be one that resulted in an increase in levels of one or more of HSPA9, HSPA5 and HSPA8 in the aged subject's skin, for example, toward the levels of these proteins in young control subjects, thereby providing a directed treatment for improving aging skin attributes.

By way of further example, the levels or amount of expression of the following subset of proteins from Table 3 (Example 1) herein: TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2; and CRYAB was found to increase in the skin of aged or old subjects versus young controls. Accordingly, an optimal treatment or therapeutic product or regimen for aging skin would be that which caused a decrease in the levels of such proteins in the skin of aged subjects, for example, toward the levels found in the skin of young control subjects. Thus, if a treatment or therapeutic product or regimen involving contact of the skin, skin tissue, or skin cells of an aged or old subject is evaluated and found to decrease the levels or amount of expression of any of these biomarker proteins relative to the control, then the treatment or therapeutic product or regimen may be identified as being effective in treating or preventing the aging of skin.

In other embodiments, changes in the levels of one or more of the proteins of Table 3 correlate with severity of one or more skin attributes, e.g., wrinkles, sagging, or pigmentation, in an aged or old subject relative to control. Thus, a treatment or therapeutic product or regimen that appropriately affected the levels of the one or more proteins in a subject's skin would be identified as being effective in treating or preventing skin aging in the subject.

### Kits

Also provided by the present invention is a kit that contains a substrate having attached thereto reagents to detect the skin biomarkers set forth in Table 3 or a subset or panel of biomarker proteins thereof. The kit can optionally contain a receptacle for a subject's skin sample and appropriate reagents for preparing the skin sample for application to the substrate. Suitable control reagents and instructions for use may also be included in the kit. The kit allows for an analysis of the skin sample at the point of service. Either the subject undergoing assessment or a medical professional can perform the assay to identify the skin condition based on the elements provided in the kit.

In another aspect, the present invention provides kits for determining or qualifying the effects of aging or of skin condition status in a subject. In particular embodiments, the kit is a diagnostic kit and comprises a substrate for collecting a biological sample, e.g., a skin sample, from the subject and means for measuring the levels of one or more protein biomarkers selected from the proteins presented in Table 3. In a particular embodiment, the levels of one or more of the HSPs presented in Table 3 are measured to determine if the levels, e.g., decreased levels, in the test skin sample are indicative of aging or of a skin condition that correlates with the HSP expression. In an embodiment, the levels of HSPA9, HSPA5 and HSPA8 are determined. The detected amounts of one or more proteins of Table 3 may correlate with aging of the skin and/or with the grading of certain skin attributes such as wrinkles, lines, pigmentation and other skin attributes.

By way of nonlimiting example, a kit may be in the form of an enzyme linked immunoassay (ELISA) kit and may include a solid support, such as a (bio)chip, microtiter plate (e.g., a 96-well plate), beads, or resin having biomarker capture reagents attached thereon. The kit may further contain a means for detecting the biomarkers, such as specific antibodies, and a secondary, antibody-signal complex such as horseradish peroxidase (HRP)-conjugated goat anti-rabbit IgG antibody and tetramethyl benzidine (TMB) as a substrate for HRP. Alternatively, the antibody may be labeled with a detectable label, such as fluorescent or chemiluminescent label or tag.

The kit for determining or qualifying the effects of aging or disease of skin may be provided as an immunochromatography strip comprising a membrane on which the antibodies are immobilized, and a means for detecting, e.g., gold particle bound antibodies, in which the membrane includes, for example, nitrocellulose (NC) membrane or polyvinylidene fluoride (PVDF), and the like, membrane. The kit may comprise a plastic plate on which a sample application pad, gold particle-bound antibodies temporally immobilized on a glass fiber filter, a nitrocellulose membrane containing immobilized bands of antibody and a secondary antibody, and an absorbent pad are positioned in a serial manner, so as to keep continuous capillary flow of sample, e.g., a solubilized skin sample or serum.

In certain embodiments, a subject's skin condition can be diagnosed and treatment determined by adding a skin sample extract or a solubilized skin sample from the subject to the kit and detecting one or more relevant protein biomarkers bound by specific antibodies coupled to a detectable compound or molecule, specifically, by a method which comprises the steps of: (i) collecting a skin sample from the patient; (ii) solubilizing the sample; (iii) adding the sample to the diagnostic kit; and, (iv) detecting the biomarker (b) bound by the detectable antibodies. In this method, the antibodies are brought into contact with the skin associated proteins from the subject's skin sample. If the biomarkers are present in the sample, the antibodies will bind to the sample, or a portion thereof. In other embodiments, a previously collected and/or previously prepared subject's sample is used in the method. In other embodiments, the sample may comprise a skin tissue sample.

The described kits can also contain a washing solution or instructions for making a washing solution, in which the combination of the capture reagents and the washing solution allows capture of the biomarkers on the solid support for subsequent detection by, e.g., antibody-based methods or mass spectrometry. In another embodiment, a kit may contain instructions for suitable operational parameters in the form of a label or separate insert. For example, the instructions may inform a consumer, clinician, or medical practitioner about how to collect a sample, how to wash the probe or the particular biomarkers to be detected, etc. In another embodiment, the kit can contain one or more containers with biomarker samples, to be used as standard(s) for comparison and/or calibration purposes.

In another embodiment, a kit may include antibodies that specifically bind one or more of the protein biomarkers of Table 3 whose levels change with the aging of skin or when associated with another type of skin condition or disorder. In an embodiment, the antibodies specifically bind heat shock proteins HSPA3, HSPA5 and HSPA8 if present in the sample. Such a kit may also contain a tape or tape material as described herein for collecting skin sample (e.g., skin tissue or cells), a set of reagents for immunochemically detecting the proteins collected on the tape and instructions for use. In an embodiment, the instructions include a description of the use of the kit, as well as the assessment criteria used to determine and rate the degree of aging of the skin sample, for example.

In another embodiment, a kit may include nucleic acid probes capable of specifically hybridizing with mRNA associated with genes encoding one or more of the biomarker proteins of Table 3 whose levels change with aging of skin, or with the presence of a skin condition or disorder. In an embodiment, the probes may hybridize with mRNA of one or more heat shock proteins, e.g., HSPA9, HSPA5 and HSPA8. The kit may also include a tape or tape material as described herein for collecting skin sample (e.g., skin tissue or cells), reagents for extracting RNA from the skin tissues collected on the tape, reagents for analyzing RNA using a known method, e.g., Northern blot, and instructions for use, which optimally include assessment criteria used to determine and rate the degree or extent of aging of the skin sample correlated with increased or decreased levels of the proteins, for example.

In another embodiment, a kit may include a pair of nucleic acid primers comprising a nucleic acid primer capable of specifically hybridizing with mRNA of one or more of the Table 3 proteins whose levels change with aging of skin, and a nucleic acid primer capable of specifically hybridizing with cDNA synthesized using the aforementioned mRNA as template. In an embodiment, the mRNA may be that of one or more heat shock proteins, e.g., HSPA9, HSPA5 and HSPA8. The kit may also include a tape or tape material as described herein for collecting skin sample (e.g., skin tissue or cells), reagents for extracting RNA from the skin tissues collected on the tape, reagents for analyzing RNA using a known method, e.g., Northern blot, and instructions for use, which optimally include assessment criteria used to determine and rate the degree or extent of aging of the skin sample correlated with alterations in levels of the proteins, for example, decreased levels of the HSPs relative to control levels.

Additional details and aspects of the present invention will be apparent from the following examples, which are not intended to be limiting.

### EXAMPLES

### Example 1

### Evaluation of Skin Aging Biomarkers in the Skin Stratum Corneum of Young Versus Old Human Subjects (Clinical Study)

### Background and Rationale of Study

The aging of human skin manifests itself in many ways, particularly on the sun-exposed areas of the body. Exposure to decades of sunlight results in a gradual structural degradation and the appearance of an assortment of unwelcome signs of photoaging, viz. dyspigmentation, wrinkles, roughness, dullness, yellowness, leathery texture and laxity. These adverse changes appear more prominently on the dorsal forearms and the face, since these parts of the body are relatively more exposed to sun during everyday activities. In the middle to later years of life, the degradation of skin increases significantly.

Some of the symptoms of aging skin are the result of degradation of collagen and elastin fibers, thinning of skin and subcutaneous adipose tissue layers. This is accompanied by dry/scaly and rough skin. Biochemical changes and changes in levels of various protein biomarkers occur during aging of skin. These biomarkers can be collected on adhesive discs from the stratum corneum and biochemically analyzed for specific expression of biomarkers, as well as specific sets of biomarkers, and the levels and alterations thereof.

This Example describes an open study to assess the skin aging phenomenon by comparing a young group of human subjects, 18-25 years of age, to an old group of subjects, 60+ years of age. This study goal was to observe, qualitatively and quantitatively, the structural and biochemical changes in skin correlated with age of the subjects.

### Study Objective

A primary objective of the study was to assess the skin aging biomarkers on dorsal forearms, inner arms and face, using adhesive discs for sampling the stratum corneum, and comparing the stratum corneum of a cohort of young subjects to that of a cohort of old subjects. A secondary objective was to show the differences in young and old skin by instrumental measurements and imaging.

### Summary of Study

A panel of 20 Caucasian female volunteers, 10 between the ages of 18 and 25 years, and 10 over the age of 60 years, forming two groups of subjects one young and one old, participated in this study. All subjects signed the Informed Consent Document and the Photography Consent Document, and underwent the recruitment procedure based on the Inclusion/Exclusion Criteria. The subjects did not use any topical product on their left dorsal forearm, left upper-inner arm and face on the day of evaluation. The following measurements/imaging were made on each subject's left dorsal forearm, left upper-inner arm and face (left and right):

1) Digital Photography (Visia-CR for face and standard photography for left dorsal forearm and left upper-inner arm), 1×5 face, 1×3 dorsal forearm (left) and 1×3 upper-inner arm (left);
2) Fluorescence Photography (fluorescence photo system), 1× dorsal forearm (left) and 1× upper-inner arm (left);
3) Reflectance spectrophotometry by a Reflectance Spectrophotometry (DRS) device, 3× readings from each site, face (left and right), dorsal forearm (left), Upper-inner arm (left), for a total of 12 readings;
4) Spectrofluorimetry by SkinSkan, 3× readings from each site, face (left and right), dorsal forearm (left), Upper-inner arm (left), a total of 12 readings; and after 20 minutes of acclimation in an Environmental Chamber, at 70° F. temperature and 40% relative humidity;
5) Hydration by Skicon, 3× readings from each site, face (left and right), dorsal forearm (left), Upper-inner arm (left), a total of 12 readings;
6) Transepidermal Water Loss (TEWL), 3× readings from each site, face (left & right), dorsal forearm (left), Upper-inner arm (left), a total of 12 readings; and
7) Stripping by D-SQUAME™ adhesive discs on four areas on each site, face, left dorsal forearm and left upperinner arm. On the face, stripping was done on two areas on the left and two areas on the right. Five strippings were obtained from each of the 4 areas on each of the three sites—the left dorsal forearm, left upper-inner arm and the face, respectively. The D-SQUAME™ strippings were stored in a deep-freezer at −20° Celsius, and were sent to the sponsor at the end of the study (all 20 subjects completed) over dry ice. The study did not include skin treatments. In the Results presented, the Young skin was compared to the Old skin.

### Candidate Study Participants

The panel of twenty (20) female subjects was selected at random from a pool of volunteers representing a local population. A pertinent medical history was obtained from each candidate.

For the study Inclusion Criteria, a candidate had to demonstrate that she was a healthy female either between 18 and 25 years of age (Young Group) or above 60 years of age (Old Group); that her left dorsal forearm, left upper-inner arm and face were generally free from any blemishes, scars, or recent sunburn/suntan; that she had a Fitzpatrick skin type between I-III; that she demonstrated an ability to follow study instructions and seemed likely to complete all requirements, and that she was able to read and understand the Informed and Photographic Consent Documents, and was willing to sign them.

For the Exclusion Criteria, a candidate was excluded from the study if any of the following was disclosed in the medical history or interview: 1) concomitant participation in another investigational drug or device study; 2) any uncontrolled systemic disease which may interfere with evaluations; 3) use of topical Retinoid on the evaluation sites within one (1) month of the study start; 4) use of excessive amounts of Vitamin A within two (2) weeks of the study start; 5) use of cosmetics, lotions, creams, and/or gels on the evaluation sites on the study day; 6) treatment with systemic corticosteroid or immunosuppressive medications within 14 days prior to the start of the study; 7) had received any type of dermatological treatment or surgery on the evaluation sites in the past two (2) months by a Cosmetic Dermatologist or Plastic Surgeon such as laser treatments, chemical peels, microdermabrasion, injections of fillers or Botox, or any treatment involving intense pulsed light or radiofrequency energy; 8) had used prescription medications, oral or topical which, in the opinion of the Investigator may interfere with the study evaluations; 9) had an incidence of skin cancer on the evaluation sites within the past one (1) year; 10) had a history of Polymorphous Light Eruption (PLE) or Solar Urticaria; or 11) had a history of uncontrolled diabetes or renal disease; had sunburn on the forearms within the past two (2) weeks or current peeling due to sunburn.

### Sites of Skin Evaluation

In the study, the number of sites for photography of the results included the dorsal forearm L; the upper-inner arm L; and the face L and R. The number of sites for SkinSkan and DRS include 1) Face R; 2) Face L; 3. Upper-inner Arm L; and 4) Dorsal Forearm L. The below Table 1 shows the numbers of readings and measurements made on each of the above-noted sites. Table 2 below shows the number of D-SQUAMES on each site.

TABLE 1

| | | IMAGING AND MEASUREMENTS | | | | | | | |
|---|---|---|---|---|---|---|---|---|---|
| | | Imaging | Imaging Arm std | Imaging Arm | | | | | |
| | | Face | camera Std + | Fluorescence | | | | | |
| Site # | Sites | Visia-CR | Xpol + //pol | camera | DRS | SkinSkan | Hydration | TEWL | D-SQUAME |
| 1 | Dorsal | | 3-images | 1-image | 3-rdngs | 3-rdngs | 3-rdngs | 3-rdngs | 20-dsq |
| | Forearm L | | | | | | | | |
| 2 | Upper-inner | | 3-images | 1-image | 3-rdngs | 3-rdngs | 3-rdngs | 3-rdngs | 20-dsq |
| | Arm L | | | | | | | | |
| 3 | Face R + L | 5-images | | | 6-rdngs | 6-rdngs | 6-rdngs | 6-rdngs | 20-dsq |
| | Totals> | 5-images | 6-images | 2-images | 12-rdngs | 12-rdngs | 12-rdngs | 12-rdngs | 60-dsq |

TABLE 2

| | Dorsal Forearms | Upper-Inner Arm | Face | | |
|---|---|---|---|---|---|
| | L | L | R | L | Totals |
| # of Areas | 4 | 4 | 2 | 2 | =12 |
| # of Dsq | 20* | 20* | 10* | 10* | =60 |

*Five D-SQUAMES from each area

### Materials and Methods Related to the Study

### D-SQUAME™ Adhesive Discs

D-SQUAME™ (CuDerm Corp. Dallas, Tex.) is a transparent, adhesive disc of 22 mm diameter. It is used to remove a layer of stratum corneum for cytology and cell morphology. The disc was pressed onto the skin site with a spring-loaded pad and then removed gently. Each disc was placed in an appropriately labeled sample collection tube provided by the sponsor. The sample tubes were stored in a freezer at a temperature of −20° Celsius.

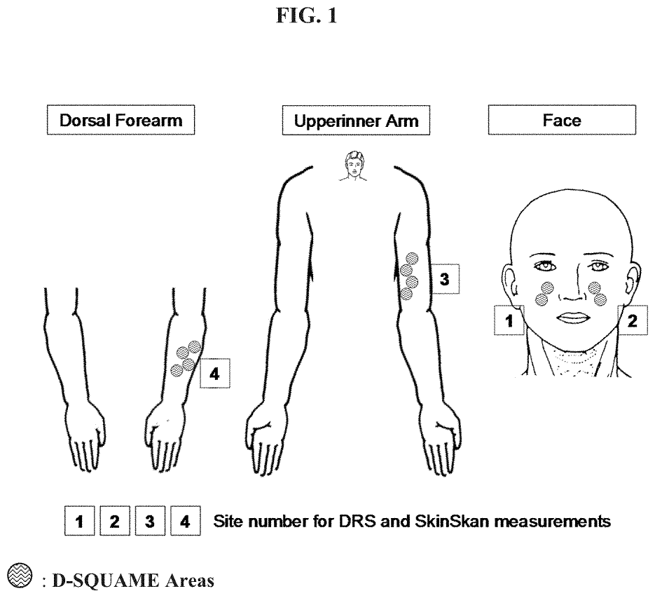

Four areas on each of the sites, i.e., dorsal forearm (left), upper-inner arm (left) were identified for the adhesive D-SQUAME™ disc stripping of stratum corneum (called “D-SQUAMES” herein). On the face, there were two areas on the left cheek and two areas on the right cheek, as shown in FIG. 1. Five D-SQUAMES were obtained from each area.

### Digital Photography

The photographs of the arm (left dorsal forearm and left upper-inner arm) were obtained using a computerized photography system (Canfield Imaging System, Fairfield, N.J.) with a 21.1-Megapixel (5616×3744 pixels) Canon EOS 5D Mark II digital camera. A Canon zoom lens, EF 24-85 mm, set at 85 mm, 1:3.5-4.5 was used with the camera. The camera+lens, along with the twin flash lamps (Canfield Intelliflash), were mounted on a Photography-Table. A height-adjustable stand was used to position the dorsal forearm horizontally in front of the camera and the photo was captured from the elbow to the wrist. A black cardboard was mounted behind the forearm. Similarly, the upper-inner arm was positioned in front of the camera and photographed. To obtain the polarized photos, linear polarizers were mounted on the flash lamps as well as on the camera lens. A parallel-polarized photo was captured when the camera and flash lamp polarizers were parallel to each other. The camera polarizer was then be rotated by a 90° angle to obtain the Cross-polarized photo.

Mirror software (Canfield Imaging Systems, NJ) was used for controlling the camera and saving the captured photos in a database. The Mirror “PhotoFile Image Management” is a data-base program which helps in storing, retrieving and exporting captured images, as well as applying attributes to distinguish between different sets of images. The “Live-View” mode was used to display the picture of the forearm, upper-inner arm or face on the computer screen in real-time within a rectangular frame with vertical and horizontal grid lines. The photography sites were aligned to the grid lines and the photos will be captured by clicking the “Capture” bar.

### Fluorescence Photography

The fluorescence photography of the left dorsal forearm and left upper-inner arm was be done using a pair of filtered flash lamps, emitting violet-blue light, and a digital camera filtered to receive green-yellow-red light. The photographic equipment consisted of a standardized camera-table unit (Canfield Clinical Systems, Cedar Grove, N.J.). The digital camera is a Nikon D90 with a 12.3 million-pixels sensor. The objective lens is a Nikkor 60-mm 2.8 AF (Nikon Inc., Melville, N.Y.). Two band-pass interference filters, emitting at a center wavelength of 417 nm (UVA-Blue, 385-430 nm) were mounted in front of two 400-Watts flash lamps (Norman, Model 404, Burbank, Calif.). The flash lamps were positioned symmetrically at the side of the camera body. A UVA-Blue cutting filter, GG475 (Schott Glass, Duryea, Pa.) was placed in front of the camera lens.

### Visia-CR

Visia-CR (Canfield Scientific, NJ) is a facial photography system that captures images under five different lighting conditions: FDA standard of visible light, modified visible light, parallel-polarized, cross-polarized and UV. For each subject, one photograph of the face was taken in the front pose, with Visia-CR saving five different photos automatically. Mirror software (Canfield Imaging Systems, N.J., USA) was used for controlling the camera and saving the captured photos in a database.

### DRS (Reflectance Spectrophotometry)

DRS is an analytical tool for the investigation of optical scattering and absorption properties of the skin when a beam of light penetrates into the skin. Briefly, the system consists of a broadband light source, a bifurcated fiber optics probe and a spectrometer. The probe contains 600 randomly mixed borosilicate fibers of 50 μm core diameter. The distal end of the probe has a total diameter of 2 mm. One proximal end of the fiber probe is coupled to a tungsten halogen light source and the other to an Ocean Optics spectrometer USB2000. Measurements were performed by placing the common end of the fiber bundle gently in contact with skin so as not to perturb the blood content. A reflectance spectrum was acquired in the range of 400-820 nm. The absorbance spectrum of the skin site was calculated as the logarithm of the ratio of the diffuse reflectance from the skin site to the diffuse reflectance from a white reflectance standard. Pigment was evaluated from the absorbance curve as the slope of the fitted straight line over the wavelength range of 620-720 nm. After the absorbance curve for the pigment absorption was corrected, the oxy-hemoglobin and deoxy-hemoglobin absorption curves were in the range of 550-580 nm, where they exhibited maxima. Three readings were taken from each site.

### SkinSkan (Spectrofluorimetry)

In vivo fluorescence spectroscopy was performed using a SPEX SkinSkan spectrofluorimeter (JY Horiba, Edison, N.J., U.S.A.). The excitation source is a Xenon arc lamp. The scanning time and UV irradiance for each wavelength that are emitted from the 200 micrometers fibers that are in contact with skin make the irradiation doses negligible; therefore, no skin reaction is expected. Measurements were performed by placing the optical fiber probe in contact with the skin site of interest. Before each set of measurements was taken, the instrument was spectrally calibrated for excitation and emission in the region of 250-650 nm. The chromatic resolution of the spectrofluorimeter was ±2 nm (provided by the manufacturer). Acquisition of excitation spectra is the preferred method of measuring in vivo skin fluorescence. Fluorescence excitation spectra permit the identification of excitation bands associated with specific emission bands. The focus of this study was in the tryptophan moieties band, with excitation band at 295 nm and emission at 340-350 nm; and the pepsin-digestible collagen cross-link band with excitation band at 335 nm and emission maximum at 380-390 nm. In order to measure these two bands, batch program were run, first scanning the “tryptophan band” and then the “collagen cross-link band.” Measurements were performed in triplicate on the tested sites.

### Environmental Chamber

Stratum corneum hydration and transepidermal water loss (TEWL) measurements were performed inside an environmental chamber where the temperature was maintained at 70° F.±1° F. and relative humidity was 40%±5%. A closed chamber with a single entry door with thick wall insulation was used for controlling the temperature and humidity. A highly precise process control unit was used to read the values of temperature and humidity sensors and to control the ambient condition by sending pulses of hot, cold and humid air, along with cycles of de-humidification.

The mixing of hot, cold and humid air was carried out outside the chamber in a sub-chamber above the ceiling. The ceiling has 56800 micro-holes, 3 mm diameter each and distributed 12 mm apart. The mixed air from above continuously filtered through these micro-holes down into the chamber to provide a uniform atmosphere inside and held the temperature and humidity constant. There were no air-blowing fans.

The inner dimensions of the chamber are: Width 301 cm, Length 355 cm and Height 213 cm. Outside the chamber there are refrigeration and heating units as well as humidity and de-humidifying units. The humidifying system consists of water mist producer (vapor injection). The water supplied to this unit comes through a two-stage laboratory water filter. The de-humidifier system consists of a large freezer coil with condenser fins, placed just outside the wall, across a screened opening. The temperature could be varied between 15° C. (59° F.) and 44° C. (111.2° F.). Relative humidity could be set between 40 and 95 percent. The temperature and humidity were continuously recorded on a chart recorder. Subjects were acclimated for between 15 and 30 minutes.

### Hydration by Skicon (Conductance)

Stratum corneum (SC) hydration was assessed by an electrical conductance measuring device using 3.5 megahertz signal (Skicon-200, I.B.S. Co. Ltd. Japan). A spring-loaded probe with concentric electrodes, was placed on the skin surface for 2 seconds. The reading shown on the LED display was printed on paper. Measurements were done in an environmental chamber at a temperature of 70° F.±1° F. and a relative humidity of 40%±5%.

### Trans-Epidermal Water Loss (TEWL)

Barrier function of skin was assessed by measuring the transepidermal water loss (TEWL) with an evaporation measuring device (Dermalab, Cortex Technology, Denmark). Measurements were made inside an environmental chamber at a temperature of 70° F.±1° F. and a relative humidity of 40%±5%. The data were collected for 60 seconds. The values in g/m²h were recorded on the case record form (CRF). Hydration and TEWL data were collected in tables. The DRS and SkinSkan data were electronic, saved as files on the computer. The photos were digital, also saved as files on the computer. Two digits were used to designate the subject number (01, 02, 03 . . . 20) for SkinSkan and DRS filenames; one digit was used to designate the clinical sites (1, 2, 3 or 4); and one digit was used to designate repletion (1, 2 or 3). For example: ‘0111’ designated: subject number one, site number 1 and reading number 1.

### Study Procedure

For the study procedure, twenty (20) female subjects participated. Ten (10) of the subjects were between the ages of 18 and 25 years and ten (10) were over the age of 60 years, forming two groups (cohorts) of subjects: one young group and one old group.

According to the study protocol, each subject arrived at the Testing lab without having applied any topical product/products that day on their left dorsal forearm, left upper-inner arm and face. After each subject signed an Informed Consent Document and a Photographic Consent Document, a medical history was obtained and each subject was screened for eligibility according to the Inclusion/Exclusion Criteria set forth above. Subjects who met the entry criteria were entered into the study and were assigned a subject number from the series 01, 02, 03 . . . 20.

Digital photography of the dorsal forearm, upper-inner arm and the face was performed using the camera set up described above. One face photo was taken in front pose by Visia-CR, followed by standard photography of left dorsal forearm and left upper-inner arm. Fluorescence photography of the left dorsal forearm and left upper-inner arm was done following the white-light digital photos, using a different photography system in the above-described “Fluorescence Photography” section. One fluorescence photo was obtained from each site. Reflectance Spectrophotometry (DRS) measurements were done on the four sites; with three readings recorded for each site. Spectrofluorimetric (SkinSkan) measurements were done on four sites, with three readings recorded for each site.

Each subject acclimated in an Environmental Chamber for 20 minutes in preparation for hydration and TEWL measurements. Inside the environmental chamber the temperature was 70° F.±1° F. and relative humidity was 40%±5%. Hydration by Skicon (conductance) was done on each of the four sites, with three readings recorded for each site. Trans-epidermal Water Loss (TEWL) measurements were done on each of the four sites, with three readings recorded for each of the sites. D-SQUAME stripping of the stratum corneum was performed at the end on all of the sites. Four areas on each of the sites, i.e. dorsal forearm (left), upper-inner arm (left), and face were identified for the adhesive D-SQUAME disc stripping of the stratum corneum. Five D-SQUAME strips were obtained, sequentially, from each of the following areas on the face: two areas on the left cheek and two areas on the right cheek (FIG. 1). The D-SQUAMES were stored in individually labeled sample collection tubes, which were placed at −20° Celsius until all samples were collected for all the subjects prior to analysis.

For the reporting of results, compiled information, including hydration and TEWL data, were forwarded to the Sponsor approximately 6 weeks after completion of the study. The digital photos of all subjects were sent to the study sponsor on a suitable medium, e.g. DVD, within approximately 6 weeks. Raw data from the DRS and SkinSkan measurements were sent to the sponsor within 2 weeks of study completion. The D-SQUAME discs were mailed over dry ice within approximately 3 days of study completion.

According to the study protocol, a subject may withdraw from the study without prejudice at any time and for any reason, but must report such reason fairly and accurately. Subjects who do not comply with the study requirements during any part of the study may, at the discretion of the Investigator, be withdrawn from further participation in the study.

Regarding adverse events, most of the measuring techniques used in this study were non-invasive, with the exception of D-SQUAME stripping, which is partially invasive. However, no adverse events were expected. Any unusual effects which occurred were noted. Skin protein profiles were compared between the young and old female subjects using proteomics analysis as described below.

### Proteomics Analysis

### In Gel Digestion

The tape strips were incubated with 50 μl of 1× loading buffer containing 50 mM DTT at 95° C. for 5 minutes, sonicated in ice-water for 5 minutes, and then centrifuged at 20,000×g for 10 minutes. The supernatant was applied to an SDS-PAGE gel and electrophoresed into the gel for 1 cm. The gel was stained with Coomassie Blue dye, and gel slices were made. Each sample in the Coomassie stained gel slices was subjected to in-gel tryptic digestion after reduction and alkylation. To this end, the gel slices were incubated at 60° C. for 30 minutes with 10 mM DTT. After cooling to room temperature, 20 mM iodoacetamide were added and the gel slices were kept in the dark for 1 hour to block free cysteines. The samples were digested by trypsin at a ratio of 1:50 (w:w; trypsin:sample) and incubated at 37° C. overnight. The digested samples containing peptides were extracted, dried under vacuum and solubilized in 5% acetonitrile, 0.1% TFA.

### LC-MS/MS

The digested samples were analyzed by nanoLC-MS/MS using a RSLC system (interfaced with a Q Exactive (ThermoFisher, San Jose, Calif.) with a nanoelectrospray ion source (Proxeon). Samples were loaded onto a self-packed 100 μm×2 cm trap that was packed with Magic C₁₈AQ, 5 μm 200 A (Michrom Bioresources Inc., Aubum, Calif.) and washed with Buffer A (0.2% formic acid) for 5 minutes with a flow rate of 5 μl/minute. The trap was brought in-line with the homemade analytical column (Magic C18AQ, 3 μm 200 A, 75 μm×50 cm) and peptides were fractionated at 300 nL/min with a multi-stepped gradient (4 to 15% Buffer B (0.16% formic acid 80% acetonitrile) for 25 minutes and 15-25% Buffer B for 65 minutes, and 25-50% Buffer B for 55 minutes). Mass spectrometry data were acquired using a data-dependent acquisition procedure with a cyclic series of a full scan acquired with a resolution of 120,000, followed by MSMS scans (30% of collision energy in the HCD cell) with resolution of 30,000 of 20 most intense ions with dynamic exclusion duration of 10 seconds.

### Data Analysis

Proteome Discoverer (ThermoFisher, version 1.4) was used to convert raw to mgf format for downstream analysis. The LC-MSMS data were searched in MUDPIT style against human database from Ensembl combined with a list of external contaminants from common Repository of Adventitious Proteins (cRAP) (maintained by the global proteome machine organization) using an in-house implementation of GPM Manager version 3.0 which uses X!Tandem (Vengeance (2015.12.15.2) the gpm.org) to assign spectral data. (Craig R. and Beavis R. C., (2004), TANDEM: Matching proteins with tandem mass spectra. Bioinformatics 20(9):1466-1467; Craig R. et al., (2004), Open source system for analyzing, validating, and storing protein identification data. J. Proteome Res. 3 (6): 1234-42; Beavis R. C., (2006), Using the global proteome machine for protein identification. Methods Mol. Biol., 328:217-28. The MudPit Style produces a merged output file which allows for a consistent grouping of spectra of all files to gene products using strict parsimony principle. Enzyme specificity was set as C-terminal to Arg (arginine) and Lys (lysine) and allowing for one missed cleavages. ±7 ppm and 20 ppm were used as tolerance for precursor (MS) and product ions (MS/MS), respectively. Carbamidomethylated cysteine was set as complete modification. N-terminal protein acetylation and oxidation of methionine were set as potential modifications. Deamidation at asparagine and glutamine, oxidation at methionine and tryptophan, dioxidation at methionine and tryptophan were allowed during model refinement stage. The statistical analysis and ratio between sample groups was done using “FDRtool” package under R environment.

### Results

Table 3 sets forth ninety-nine proteins (biomarker proteins) identified as being differentially expressed in skin samples of the young subjects tested and skin samples of the old subjects tested. Also presented in the Table is the fold-change in each of the proteins identified as differentially expressed in skin of young versus (vs) old subjects.

TABLE 3

| Protein name | Fold change (young vs old) |
|---|---|
| GLUD1 | 12.00 |
| DDX3X | 9.80 |
| ERGIC1 | 8.00 |
| NIT2 | 7.50 |
| PSMC5 | 7.00 |
| TSG101 | 7.00 |
| UQCRFS1 | 6.50 |
| ZG16B | 6.00 |
| RAB1A | 6.00 |
| PLA2G4E | 6.00 |
| GPD2 | 5.67 |
| AADAC | 5.50 |
| ATP6V1B2 | 5.50 |
| PHB | 5.43 |
| NAPRT | 5.29 |
| RAB1B | 5.29 |
| SULT2B1 | 5.13 |
| CARS | 5.00 |
| HSPA9 | 4.80 |
| ACTR2 | 4.22 |
| EIF2S1 | 4.00 |
| RPL22 | 4.00 |
| CAP1 | 3.80 |
| ATP6V1E1 | 3.75 |
| RTN3 | 3.50 |
| DYNLL2 | 3.50 |
| PHB2 | 3.43 |
| ITPA | 3.33 |
| IST1 | 3.17 |
| UQCRC2 | 3.17 |
| FASN | 3.14 |
| LMNB2 | 3.13 |
| MY018A | 3.00 |
| ATP6V1A | 3.00 |
| ACOX1 | 2.97 |
| RPN2 | 2.93 |
| RAB7A | 2.80 |
| EIF4A1 | 2.75 |
| ALDH2 | 2.69 |
| ACTR3 | 2.68 |
| ACADVL | 2.63 |
| KRT73 | 2.44 |
| ANXA5 | 2.39 |
| HNRNPH2 | 2.20 |
| VCP | 2.18 |
| MYH14 | 2.15 |
| ANXA4 | 2.03 |
| PDCD6 | 2.00 |
| HSPE1 | 2.00 |
| IDE | 1.97 |
| CKAP4 | 1.97 |
| PIP | 1.93 |
| CAPZB | 1.88 |
| ASPRV1 | 1.85 |
| MT-CO2 | 1.85 |
| CAPG | 1.82 |
| RAB14 | 1.77 |
| PTGS1 | 1.77 |
| POF1B | 1.76 |
| HSPA5 | 1.76 |
| TGM5 | 1.76 |
| HSPA8 | 1.74 |
| LGALS7B | 1.74 |
| HSPD1 | 1.73 |
| LMNA | 1.73 |
| CALML5 | 1.70 |
| HPSE | 1.60 |
| TMED9 | 1.58 |
| ACPP | 1.55 |
| FUCA1 | 1.50 |
| HEBP2 | 1.50 |
| S100A11 | 1.47 |
| RPS27A | 1.43 |
| IL1RN | 1.36 |
| CKMT1B | 1.30 |
| CTSD | 1.25 |
| TECR | −1.21 |
| KRT10 | −1.23 |
| HEXB | −1.23 |
| BLMH | −1.26 |
| CAT | −1.32 |
| SYLP1 | −1.34 |
| AGA | −1.39 |
| CTSH | −1.39 |
| STS | −1.41 |
| C11orf54 | −1.48 |
| RNASE7 | −1.55 |
| IL37 | −1.67 |
| KPRP | −1.83 |
| KRT6B | −1.86 |
| KRT75 | −2.30 |
| NRM | −3.00 |
| LGALS3BP | −3.57 |
| APOE | −3.67 |
| FAM175B | −4.50 |
| CDA | −6.00 |
| KRT76 | −9.00 |
| KRTAP13-2 | −10.00 |
| CRYAB | −10.00 |

The relative levels of the proteins shown in Table 3 are useful for determining, diagnosing, or prognosing a skin condition using a skin sample of a subject undergoing testing. As will be appreciated by the skilled practitioner in the art, the proteins in Table 3 include Glutamate dehydrogenase 1 (GLUD1); ATP-dependent RNA helicase DDX3X enzyme encoded by the human DDX3X gene (DDX3X); Endoplasmic Reticulum-Golgi Intermediate Compartment 1 (ERGIC1); Nitrilase Family Member 2 (having omega-amidase activity; (NIT2); 26S protease regulatory subunit 8 or 26S proteasome AAA-ATPase subunit Rpt6 (PSMC5); Tumor susceptibility gene 101 protein (TSG101); Ubiquinol-cytochrome c reductase, Rieske iron-sulfur polypeptide 1 (UQCRFS1); Zymogen Granule Protein 16B (ZG16B); Ras-related protein Rab-1A, (GTP binding protein), (RAB1A); Phospholipase A2 Group IVE protein (PLA2G4E); Glycerol-3-Phosphate Dehydrogenase 2 (GPD2); Arylacetamide deacetylase (AADAC); ATPase, H+ transporting, lysosomal 56/58 kDa, V1 subunit B2 protein (ATP6V1B2); Prohibitin (PHB); Nicotinate phosphoribosyltransferase (NAPRT); Ras-related protein Rab-1B (RAB1B); Sulfotransferase Family 2B Member 1 (SULT2B1); Cysteine-tRNA ligase, cytoplasmic, an Aminoacyl-tRNA synthase (CARS); Heat Shock 70 kDa Protein 9 (Mortalin) (HSPA9); Actin-related protein 2 (ACTR2); Eukaryotic translation initiation factor 2 subunit 1 (eIF2a), (EIF2S1); 60S ribosomal protein L22 (RPL22); Adenylate Cyclase Associated Protein 1 (CAP1); V-type proton ATPase subunit E 1 enzyme (ATP6V1E1); Reticulon-3 (RTN3); Dynein light chain 2 (DYNLL2); Prohibitin 2 (PHB2); Inosine Triphosphate Pyrophosphatase (ITPA); Vacuolar protein sorting-associated protein IST1 (IST1); Ubiquinol-Cytochrome C Reductase Core Protein II (UQCRC2); Fatty acid synthase (FASN); Lamin B2 (LMNB2); Myosin-XVIIIa protein (MYO18A); V-type proton ATPase catalytic subunit A protein (ATP6V1A); Peroxisomal acyl-coenzyme A oxidase 1 (ACOX1); Dolichyl-diphosphooligosaccharide-protein glycosyltransferase subunit 2/Ribophorin 2 (RPN2); Ras-related protein Rab-7a (RAB7A); Eukaryotic initiation factor 4A-I (EIF4A1); Aldehyde dehydrogenase 2 (ALDH2); Actin-related protein 3 (ACTR3); Acyl-CoA dehydrogenase, Very long-chain, (ACADVL); Keratin 73 (KRT73); Annexin A5 (ANXA5); Heterogeneous nuclear ribonucleoprotein H2 (HNRNPH2); Valosin Containing Protein (VCP); Myosin Heavy Chain 14 (MYH14); Annexin A4 (ANXA4); Programmed cell death protein 6 (PDCD6); Heat Shock Protein Family E (Hsp10) Member 1 (HSPE1); Insulin Degrading Enzyme (IDE); Cytoskeleton Associated Protein 4 (CKAP4); Prolactin-inducible protein (PIP); F-actin-capping protein subunit beta protein (CAPZβ); Aspartic Peptidase, Retroviral-like 1 (ASPRV1); Cytochrome c oxidase subunit II (MT-CO2); capping protein (actin filament), gelsolin-like (CAPG); Ras-related protein Rab-14 (RAB14); Prostaglandin-Endoperoxide Synthase 1 (PTGS1); Premature Ovarian Failure, 1B protein (POF1B); Heat shock protein A5 aka Heat Shock Protein Family A (HSP70) Member 5 (HSPA5); Transglutaminase 5 protein (TGM5); Heat shock protein A8, aka Heat Shock Protein Family A (HSP70) Member 8 (HSPA8); Galectin 7 (LGALS7B); Heat shock protein D1 aka Heat Shock Protein Family D (Hsp60) Member 1 (HSPD1); Lamin A/C (LMNA); Calmodulin-like protein 5 (CALML5); Heparanase (HPSE); transmembrane emp24 protein transport domain containing 9 (TMED9); Acid Phosphatase, Prostate (ACPP); Fucosidase, Alpha-L-1 (FUCA1); Heme binding protein 2 (HEBP2); 5100 Calcium Binding Protein A11) (S100A11); 40S ribosomal protein S27a (RPS27A); Interleukin 1 Receptor Antagonist (IL1RN); Creatinine Kinase, Mitochondrial 1B (CKMT1B); Cathepsin D (CTSD); Trans-2,3-enoyl-CoA reductase (TECR); Keratin, type I cytoskeletal 10/cytokeratin-10 (CK-10)/keratin-10 (K10) (KRT10); N-acetyl-beta-glucosaminidase subunit beta/Hexosaminidase subunit B (HEXB); Bleomycin Hydrolase (BLMH); Chloramphenicol acetyltransferase (CAT); Synaptophysin-like protein 1 (SYPL1); Aspartylglucosaminidase (AGA); Cathepsin H (CTSH); Steroid sulfatase protein (STS); Enzyme encoded by Chromosome 11 Open Reading Frame 54 (C11orf54); Ribonuclease 7 protein (RNASE7); Interleukin 37 (IL37); Keratinocyte proline-rich protein (KPRP); Keratin 6B protein (KRT6B); Keratin 75 protein (KRT75); Nurim/Nuclear inner membrane protein (NRM); Lectin Galactoside-binding Soluble 3 Binding Protein (LGALS3BP); Apolipoprotein E (APOE); Family With Sequence Similarity 175 Member B protein (FAM175B); (CDA); (KRT76); (KRTAP13-2); and (CRYAB).

The proteins in Table 3, or a subset of the proteins in the Table, can be used to identify, determine, diagnose, or prognose a skin condition. As a particular nonlimiting example, members of the heat shock protein (HSP70) family, namely, HSPA9, HSPA5 and HSPA8, embrace a subset of proteins whose levels in a subject's skin can be used to provide a treatment for the subject. More specifically, a fold change in the levels of this smaller group of HSPs not only correlated with the age of the subject but also correlated with visible or tactile attributes of a skin condition within a narrow age range.

### Example 2

### Assessment of Correlation Between Protein Biomarkers and Skin Attributes

For this analysis, 30 subjects aged 35-45 years were recruited for a protein biomarker validation study. Tape strips were applied and removed from the cheek areas of the subjects and analyzed for various protein biomarkers as described supra. Before taking the tape strips, the subjects were asked several questions regarding their skin. The questions asked were as follows: 1) Do you think you have sensitive skin?; 2) Do you think you have more or less wrinkles for your age?; 3) Do you consider your skin to be oily, dry, or normal?; and 4) Do you think your skin tone is normal for your age?. The photos of the faces of the subjects were also graded (with a scale from 0-10, with 10 as the most and 0 as the least) by a dermatologist for the following visible or tactile attributes: fine wrinkles, coarse wrinkles, mottled pigmentation, discreet pigmentation, erythema and blotchiness. The correlation between 1) protein biomarkers from the tape strip samples and skin attributes and 2) protein biomarkers and self-perceived skin conditions were calculated using Pearson-correlation coefficient.

For the protein biomarkers, correlations were found between 1) biomarkers and derm grading and 2) biomarkers and self-perceived skin conditions. It was found that detection of levels of the protein biomarkers HSPA5 and CAPZB in the subjects' skin samples correlated well with self-perceived wrinkles by the subjects (FIGS. 2A and 2B). In addition, it was found that the ratio of IL1RA:1a proteins in the subjects' skin samples correlated well with self-perceived skin sensitivity by the subjects (FIG. 2C).

### Example 3

ELISA Measurements with HSP70 Inducing Agents

Normal human dermal fibroblasts (Cascade Biologics) were seeded into four wells of a six well plate (5.0×10⁵ cells/well) and cultured overnight. The following day, cells were replenished with fresh media and test compositions were added. The test compositions including their control for each formulation:

a) control 1 (50:50 EtOH:H₂O);

b) 0.1% (w/w) Tiliacora triandra extract (extraction medium: ethanol:water in a volume ratio of 80:20);

c) 0.01% (w/w) ethanolic Tiliacora triandra extract;

d) control 2 (H₂O)

e) 0.1% (w/w) aqueous Portulaca oleracera extract;

f) 0.01% (w/w) aqueous Portulaca oleracera extract;

g) control 3 (EtOH);

h) 0.001% cannabidiol (CBD; 99% ethanolic extract);

h) control 4 (dimethylsulfoxide (DMSO))

f) 0.01% (w/w) Phytol;

g) 0.001% (w/w) Phytol; and

h) 0.01% (w/w) Arnica montana extract (Positive Control).

The active ingredient was formulated in one of the four controls as indicated. Following 24 hours of treatment, the cells were lysed and the HSP70 protein level was measured using an ELISA kit (available from Enzo Life Sciences). Cells were lysed using the lysis buffer provided with the kit. The amount of proteins for each sample was calculated using the standard curve.

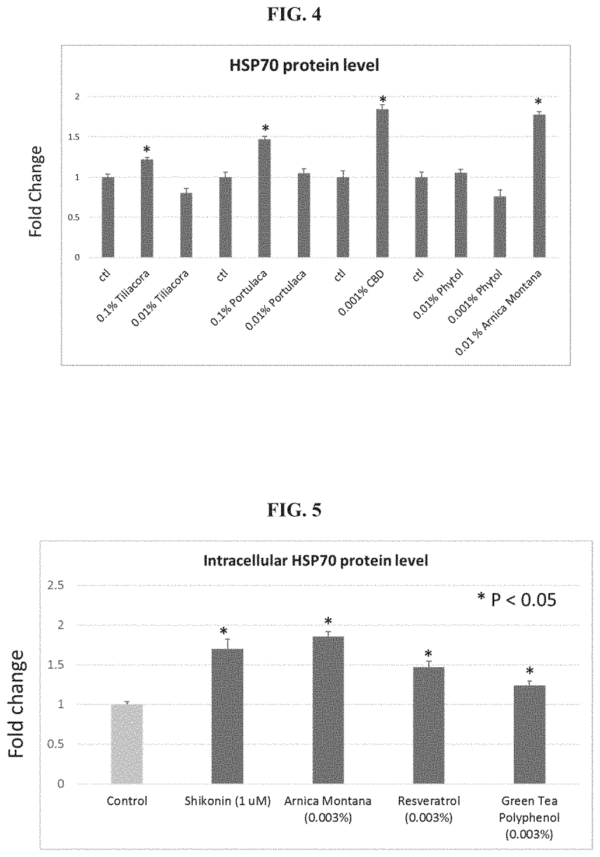

FIG. 4 shows the fold change of protein level expression in each sample as compared to its control as determined with the ELISA. The assay was performed in triplicate for each sample and the values represent mean±S.D. (standard deviation). Samples labeled with “*” indicate a p value compared to the control of less than 0.05. As can be seen, compositions comprising 0.1% Tiliacora triandra, 0.1% Portulaca oleracera and 0.01% CBD each were able to stimulate HSP70 protein level (by 122%, 150%, and 189%, respectively) in HDF cells.

Similar experimentation on other HSP70 inducers was performed using an HSP70 ELISA kit (available from Abcam; cat. # ab133060). Briefly, normal human dermal fibroblasts (Cascade Biologics) were seeded into four wells of a six well plate (5.0×10⁵ cells/well) and cultured overnight. The following day, cells were replenished with fresh media and test compositions were added. The test compositions including their control for each formulation:

a) control (50:50 EtOH:H₂O);

b) 1 μM shikonin;

c) 0.003% Arnica montana extract;

d) 0.003% resveratrol; and

e) 0.003% green tea polyphenol.

The active ingredient was formulated in the ethanol:water control. Following 24 hours of treatment, the cells were lysed and the HSP70 protein level was measured using an ELISA kit (available from Abcam). Cells were lysed using the lysis buffer provided with the kit. The amount of proteins for each sample was calculated using the standard curve.

FIG. 5 shows the fold change of protein level expression in each sample as compared to its control as determined with the ELISA. The assay was performed in triplicate for each sample and the values represent mean±S.D. (standard deviation). Samples labeled with “*” indicate a p value compared to the control of less than 0.05. As can be seen, compositions comprising 1 μM shikonin, 0.003% resveratrol, and 0.003% green tea polyphenol each stimulate HSP70 protein level (by 170%, 147%, and 124%, respectively, as compared to control) in HDF cells.

### Example 4

Experiments with 3-D Skin Tissue Equivalents

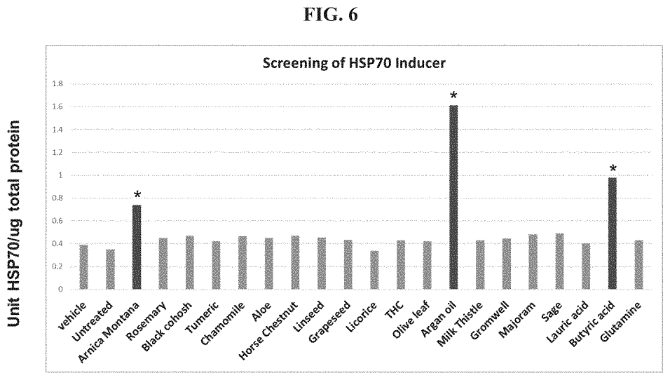

A series of experiments was conducted to assess the effects of various treatments with HSP70 inducers on full thickness 3D skin cultures. Human 3D skin (MatTek, Mass.) were cultured following the manufacturer's instructions overnight. The following day, tissues were replenished with fresh media and then compositions comprising various potential actives were added. The actives measured are shown in FIG. 6. Following 24 hours of treatment, the tissues were lysed and the HSP70 protein level was measured using an ELISA kit (available from Abcam). The amount of the proteins was calculated according to the standard curve. For these measurements, the test materials were formulated in control at 1% by weight of the composition or at 0.04% by weight of the composition (Arnica montana, rosemary, black cohosh, turmeric, chamomile, aloe, horse chestnut, linseed, grapeseed, argan oil, gromwell, marjoram, sage, lauric acid, butyric acid) or they were formulated in control at 0.04% by weight of the composition (licorice, tetrahydrocurcumin (THC), olive leaf, milk thistle, glutamate).

The results are shown in FIG. 6 as the measured amount of HSP70 proteins/μg total protein for each sample. Samples labeled with “*” indicate a p value compared to the control of less than 0.05. As can be seen, 1% argan oil and 1% butyric acid each stimulate HSP70 protein levels (by 408% and 248%, respectively, as compared to control) in skin 3-D tissues.

### Specific Embodiments

Enumerated specific embodiments of the disclosure are described below.

### Specific Embodiment 1

A method of assessing the degree of skin aging or of an age-associated skin attribute in a subject, the method comprising:

assaying in a skin sample obtained from the subject the levels of a subset of skin-associated biomarkers set forth in Table 3 relative to control levels;

measuring an alteration in the levels of subset of skin biomarkers set forth in Table 3 relative to control levels; and

assessing degree of skin aging or an age-associated skin attribute in the subject based on the measured alteration comprising increased or decreased levels of the subset of skin biomarkers in Table 3 relative to the control levels.

### Specific Embodiment 2

The method according to specific embodiment 1, wherein the subset of skin-associated biomarkers is one or more proteins set forth in Table 3.

### Specific Embodiment 3

The method according to specific embodiment 1, wherein the subset of skin-associated biomarkers is two or more proteins set forth in Table 3.

### Specific Embodiment 4

The method according to any one of specific embodiments 1 to 3, wherein the alteration comprises a decrease in the levels of a subset of skin-associated biomarker proteins comprising GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD set forth in Table 3 in the skin of the subject relative to the control, and wherein a deficiency in the levels of the subset of skin-associated biomarker proteins in the subject's skin indicates skin aging in the subject.

### Specific Embodiment 5

The method according to any one of specific embodiments 1 to 4, further comprising administering to the subject a treatment regimen or product which increases the levels of one or more of the skin-associated biomarker proteins in the skin of the subject, thereby treating aging skin in the subject.

### Specific Embodiment 6

The method according to any one of specific embodiments 1 to 3, wherein the alteration comprises an increase in the levels of a subset of skin-associated biomarker proteins comprising TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB set forth in Table 3 in the skin of the subject relative to the control, and wherein an increase in the levels of the subset of skin-associated biomarker proteins in the subject's skin indicates skin aging in the subject.

### Specific Embodiment 7

The method according to any one of specific embodiments 1 to 3 or 6, further comprising administering to the subject a treatment regimen or product which decreases the levels of one or more of the skin-associated biomarker proteins in the skin of the subject, thereby treating aging skin in the subject.

### Specific Embodiment 8

The method according to any one of specific embodiments 1 to 7, wherein the subset of skin-associated biomarkers comprise heat shock proteins (HSPs) set forth in Table 3.

### Specific Embodiment 9

The method according to specific embodiment 8, wherein the HSPs comprise HSPA9, HSPA5 and HSPA8.

### Specific Embodiment 10

The method according to any one of specific embodiments 1 to 3, wherein, if an increase in the levels of the one or more biomarkers of Table 3 is identified in the subject's skin sample relative to control levels, a treatment which decreases the levels of said biomarkers toward control levels is administered to the subject and/or is recommended for administration to the subject to treat or prevent skin aging or a skin attribute in the subject.

### Specific Embodiment 11

The method according to any one of specific embodiments 1 to 3, wherein, if a decrease in the levels of the one or more biomarkers of Table 3 is identified in the subject's skin sample, a treatment which increases the levels of said biomarkers toward control levels is administered to the subject and/or is recommended for administration to the subject to treat or prevent skin aging or a skin attribute in the subject.

### Specific Embodiment 12

The method according to any one of specific embodiments 1 to 11, wherein the skin sample is a stratum corneum sample.

### Specific Embodiment 13

The method according to any one of specific embodiments 1 to 12, wherein the skin sample is from a subject who is 60-plus years of age and the control comprises one or more subjects of 18 to 20 years of age.

### Specific Embodiment 14

The method according to any one of specific embodiments 1 to 13, wherein skin aging or attribute comprises one or more of wrinkles, fine lines, creases, folds, sagging, fragility, aberrant pigmentation, dull complexion, or weakness.

### Specific Embodiment 15

A method of treating a skin condition or disorder in a subject, the method comprising:

(a) measuring the levels of one or more skin biomarker proteins in Table 3 in a skin sample obtained from the subject;

(b) determining the levels of the one or more skin biomarker proteins in step (a) relative to levels of the same protein biomarkers in a skin sample from control subjects; wherein increased or decreased levels of the one or more skin biomarker proteins relative to the control levels identifies the skin condition or disorder in the subject; and

(c) administering to the subject a treatment that targets the skin condition or disorder associated with the increased or decreased levels of the one or more skin biomarker proteins, thereby treating the subject's skin condition or disorder.

### Specific Embodiment 16

The method according to specific embodiment 15, wherein the skin sample is a stratum corneum sample.

### Specific Embodiment 17

The method according to specific embodiment 15 or specific embodiment 16, wherein the treatment comprises increasing the levels of a subset of skin-associated biomarker proteins comprising GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD as set forth in Table 3 in the subject's skin toward control levels, thereby treating the skin condition or disorder in the subject.

### Specific Embodiment 18

The method according to specific embodiment 17, wherein the treatment comprises increasing the levels of one or more of HSPA9, HSPA5 and HSPA8 in the subject's skin.

### Specific Embodiment 19

The method according to specific embodiment 15 or specific embodiment 16, wherein the treatment comprises decreasing the levels of a subset of skin-associated biomarker proteins comprising TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB as set forth in Table 3 in the subject's skin toward control levels, thereby treating the skin condition or disorder in the subject.

### Specific Embodiment 20

The method according to any one of specific embodiments 15 to 19, wherein the subject is 60-plus years of age.

### Specific Embodiment 21

The method according to specific embodiment 20, wherein the control is a young subject having skin in the absence of the skin condition or disorder.

### Specific Embodiment 22

A method of predicting the likelihood that a subject has, or is at risk for developing, an adverse skin attribute or aging skin, the method comprising:

(a) measuring the levels of a subset of protein biomarkers from Table 3 in a skin sample obtained from the subject;

(b) identifying the levels of the subset of protein biomarkers in step (a) relative to levels from a suitable control; and

(c) predicting that subject has, or is at risk of developing, an adverse skin attribute or aging skin by assessing increased or decreased levels of the subset of protein biomarkers relative to the control, which increased or decreased levels correlate with the adverse skin attribute or aging skin.

### Specific Embodiment 23

The method according to specific embodiment 22, wherein the adverse skin attribute is selected from aging skin, mottled pigmentation, wrinkles, or a combination thereof, in the skin.

### Specific Embodiment 24

The method according to specific embodiment 22 or specific embodiment 23, wherein increased levels of the subset of protein biomarkers TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB as set forth in Table 3 relative to control levels correlates with the adverse skin attribute or skin aging.

### Specific Embodiment 25

The method according to specific embodiment 22 or specific embodiment 23, wherein decreased levels of the subset of protein biomarkers GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD as set forth in Table 3 relative to control levels correlates with the adverse skin attribute or skin aging.

### Specific Embodiment 26

The method according to specific embodiment 25, wherein the subset of protein biomarkers of Table 3 comprises HSPA9, HSPA5 and HSPA8.

### Specific Embodiment 27

The method according to specific embodiment 26, wherein the subset of heat shock proteins is HSPA5.

### Specific Embodiment 28

A method of treating aging skin in a subject in need thereof, the method comprising:

(a) measuring the levels of a subset of protein biomarkers in Table 3 in a skin sample obtained from the subject;

(b) identifying the levels of the subset of protein biomarkers in step (a) relative to levels of the same proteins in a skin sample from control subjects; wherein decreased levels of the subset of protein biomarkers in the subject's sample relative to control levels identifies aging skin in the subject; and

(c) administering to the subject identified as having aging skin a treatment that increases the levels of the subset of protein biomarkers in the subject's skin, thereby treating aging skin in the subject.

### Specific Embodiment 29

The method according to specific embodiment 28, wherein the subject in need is 60-plus years of age and the control comprises one or more subjects of 18-20 years of age.

### Specific Embodiment 30

The method according to specific embodiment 28 or specific embodiment 29, wherein the subset of the protein biomarkers in Table 3 comprises one or more of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD.

### Specific Embodiment 31

The method according to specific embodiment 30, wherein the subset of the protein biomarkers in Table 3 comprises HSPA9, HSPA5, HSPA8, or a combination thereof.

### Specific Embodiment 32

A method of treating aging skin in a subject in need thereof, the method comprising:

(a) measuring the levels of a subset of protein biomarkers in Table 3 in a skin sample obtained from the subject;

(b) identifying the levels of the one or more protein biomarkers in step (a) relative to levels of the same proteins in a skin sample from control subjects; wherein increased levels of the subset of protein biomarkers in the subject's sample relative to control levels identifies aging skin in the subject; and

(c) administering to the subject identified as having aging skin in step (b) a treatment that decreases the levels of the subset of protein biomarkers in the subject's skin, thereby treating aging skin in the subject.

### Specific Embodiment 33

The method according to specific embodiment 32, wherein the subject is 60-plus years of age and the control comprises one or more subjects of 18-20 years of age.

### Specific Embodiment 34

The method according to specific embodiment 32 or specific embodiment 33, wherein the subset of the protein biomarkers in Table 3 comprises one or more of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

### Specific Embodiment 35

A method of determining a treatment for aging or aged skin or a symptom thereof in a subject in need thereof, the method comprising:

assaying in a skin sample obtained from the subject levels of a subset of the skin biomarkers set forth in Table 3 relative to control levels;

administering to the subject a treatment that changes the levels of the subset of the biomarkers in Table 3 associated with aging or aged skin toward levels of the same subset of biomarkers in skin of controls, if an increase or a decrease in the assayed levels of the subset of skin biomarkers in Table 3 relative to the control levels indicates aging or aged skin or a symptom thereof in the subject.

### Specific Embodiment 36

A method of identifying a substance that modulates biomarkers associated with skin aging and/or a skin attribute, the method comprising:

contacting a skin sample to be tested with a candidate substance under suitable conditions for a predetermined period of time;

quantifying the levels of a subset of protein biomarkers of Table 3 from the contacted skin sample, wherein levels of the subset of proteins change with skin aging and/or a skin attribute;

determining the levels of the proteins of the contacted skin sample relative to the levels of a control to evaluate the effect of the substance on the levels of the subset of proteins from the sample; and

identifying the substance that modulates the levels of the biomarkers associated with skin aging and/or the skin attribute in the skin sample relative to the control.

### Specific Embodiment 37

The method according to specific embodiment 36, wherein the skin aging and/or a skin attribute comprises wrinkles, lines, creases, abnormal pigmentation, sagging, or weakness of the skin.

### Specific Embodiment 38

The method according to specific embodiment 36 or specific embodiment 37, wherein the subset of proteins comprises one or more of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD.

### Specific Embodiment 39

The method according to specific embodiment 38, wherein the subset of proteins comprises one or more of HSPA9, HSPA5 and HSPA8

### Specific Embodiment 40

The method according to any one of specific embodiments 37 to 39, wherein the levels of the subset of proteins decrease with aging skin.

### Specific Embodiment 41

The method according to specific embodiment 36 or specific embodiment 37, wherein the subset of proteins comprises one or more of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

### Specific Embodiment 42

The method according to specific embodiment 41, wherein the levels of the subset of proteins increase with aging skin.

### Specific Embodiment 43

A kit for determining skin aging or skin attribute status in a human subject, the kit comprising:
(a) a substrate for collecting a skin sample from the subject; and
(b) means for quantifying the levels of the protein biomarkers of Table 3 relative to control levels.

### Specific Embodiment 44

A kit for determining skin aging or skin attribute status in a human subject, the kit comprising:
(a) a substrate for collecting a skin sample from the subject;
(b) means for extracting one or more protein biomarkers of Table 3 from said substrate; and
(c) means for analyzing and/or quantifying the levels of the one or more extracted protein biomarkers from the subject relative to control levels.

### Specific Embodiment 45

The kit of specific embodiment 43 or specific embodiment 44, comprising means for analyzing and/or quantifying the levels of a subset of protein biomarkers selected from the group consisting of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD.

### Specific Embodiment 46

The kit of specific embodiment 45, further comprising means for analyzing and/or quantifying the levels of a subset of protein biomarkers selected from the group consisting of HSPA9, HSPA5, HSPA8 and a combination thereof.

### Specific Embodiment 47

The kit of specific embodiment 43 or specific embodiment 44, comprising means for analyzing and/or quantifying the levels of a subset of protein biomarkers selected from the group consisting of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

All publications cited herein are hereby incorporated by reference in their entireties, including all published patent applications, and issued patents, as well as journal articles, books, manuals. In addition, the meaning of certain terms and phrases employed in the specification, examples, and appended claims are provided. The definitions are not meant to be limiting in nature and serve to provide a clearer understanding of certain aspects of the present invention.

1. A method of treating a skin condition in a subject, the method comprising:
(a) measuring the levels of one or more skin biomarker proteins in a skin sample obtained from the subject, wherein said skin biomarker protein is selected from the group consisting of: GLUD1; DDX3X; ERGIC1; NIT2; PSMC5; TSG101; UQCRFS1; ZG16B; RAB1A; PLA2G4E; GPD2; AADAC; ATP6V1B2; PHB; NAPRT; RAB1B; SULT2B1; CARS; HSPA9; ACTR2; EIF2S1; RPL22; CAP1; ATP6V1E1; RTN3; DYNLL2; PHB2; ITPA; IST1; UQCRC2; FASN; LMNB2; MY018A; ATP6V1A; ACOX1; RPN2; RAB7A; EIF4A1; ALDH2; ACTR3; ACADVL; KRT73; ANXA5; HNRNPH2; VCP; MYH14; ANXA4; PDCD6; HSPE1; IDE; CKAP4; PIP; CAPZB; ASPRV1; MT-CO2; CAPG; RAB14; PTGS1; POF1B; HSPA5; TGM5; HSPA8; LGALS7B; HSPD1; LMNA; CALML5; HPSE; TMED9; ACPP; FUCA1; HEBP2; S100A11; RPS27A; IL1RN; CKMT1B; CTSD; TECR; KRT10; HEXB; BLMH; CAT; SYLP1; AGA; CTSH; STS; C11orf54; RNASE7; IL37; KPRP; KRT6B; KRT75; NRM; LGALS3BP; APOE; FAM175B; CDA; KRT76; KRTAP13-2; and CRYAB;
(b) determining the levels of the one or more skin biomarker proteins in step (a) relative to levels of the same biomarker proteins in a skin sample from control subjects; wherein increased or decreased levels of the one or more skin biomarker proteins relative to the control levels identifies the skin condition or disorder in the subject; and
(c) administering to the subject a treatment that targets the skin condition associated with the increased or decreased levels of the one or more skin biomarker proteins, thereby treating the subject's skin condition.

2. The method according to claim 1, wherein the skin sample is a stratum corneum sample.

3. The method according to claim 1, wherein the treatment comprises increasing the levels of a subset of skin-associated biomarker proteins comprising GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD in the subject's skin toward control levels, thereby treating the skin condition in the subject.

4. The method according to claim 1, wherein the treatment comprises increasing the levels of one or more of HSPA9, HSPA5 and HSPA8 in the subject's skin.

5. The method according to claim 1, wherein the treatment comprises decreasing the levels of a subset of skin-associated biomarker proteins comprising TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB in the subject's skin toward control levels, thereby treating the skin condition in the subject.

6. The method according to claim 1, wherein the subject is 60-plus years of age.

7. The method according to claim 1, wherein the control is a young subject having skin in the absence of the skin condition.

8. A method of treating aging skin in a subject in need thereof, the method comprising:
(a) measuring the levels of a subset of one or more biomarker proteins in a skin sample obtained from the subject, wherein said biomarker protein is selected from the group consisting of: GLUD1; DDX3X; ERGIC1; NIT2; PSMC5; TSG101; UQCRFS1; ZG16B; RAB1A; PLA2G4E; GPD2; AADAC; ATP6V1B2; PHB; NAPRT; RAB1B; SULT2B1; CARS; HSPA9; ACTR2; EIF2S1; RPL22; CAP1; ATP6V1E1; RTN3; DYNLL2; PHB2; ITPA; IST1; UQCRC2; FASN; LMNB2; MY018A; ATP6V1A; ACOX1; RPN2; RAB7A; EIF4A1; ALDH2; ACTR3; ACADVL; KRT73; ANXA5; HNRNPH2; VCP; MYH14; ANXA4; PDCD6; HSPE1; IDE; CKAP4; PIP; CAPZB; ASPRV1; MT-CO2; CAPG; RAB14; PTGS1; POF1B; HSPA5; TGM5; HSPA8; LGALS7B; HSPD1; LMNA; CALML5; HPSE; TMED9; ACPP; FUCA1; HEBP2; S100A11; RPS27A; IL1RN; CKMT1B; CTSD; TECR; KRT10; HEXB; BLMH; CAT; SYLP1; AGA; CTSH; STS; C11orf54; RNASE7; IL37; KPRP; KRT6B; KRT75; NRM; LGALS3BP; APOE; FAM175B; CDA; KRT76; KRTAP13-2; and CRYAB;
(b) identifying the levels of the subset of one or more biomarker proteins in step (a) relative to levels of the same proteins in a skin sample from control subjects; wherein decreased levels of the subset of one or more biomarker proteins in the subject's sample relative to control levels identifies aging skin in the subject; and
(c) administering to the subject identified as having aging skin a treatment that increases the levels of the subset of one or more biomarker proteins in the subject's skin, thereby treating aging skin in the subject.

9. A method of treating aging skin in a subject in need thereof, the method comprising:
(a) measuring the levels of a subset of one or more biomarker proteins in a skin sample obtained from the subject, wherein said biomarker protein is selected from the group consisting of: GLUD1; DDX3X; ERGIC1; NIT2; PSMC5; TSG101; UQCRFS1; ZG16B; RAB1A; PLA2G4E; GPD2; AADAC; ATP6V1B2; PHB; NAPRT; RAB1B; SULT2B1; CARS; HSPA9; ACTR2; EIF2S1; RPL22; CAP1; ATP6V1E1; RTN3; DYNLL2; PHB2; ITPA; IST1; UQCRC2; FASN; LMNB2; MY018A; ATP6V1A; ACOX1; RPN2; RAB7A; EIF4A1; ALDH2; ACTR3; ACADVL; KRT73; ANXA5; HNRNPH2; VCP; MYH14; ANXA4; PDCD6; HSPE1; IDE; CKAP4; PIP; CAPZB; ASPRV1; MT-CO2; CAPG; RAB14; PTGS1; POF1B; HSPA5; TGM5; HSPA8; LGALS7B; HSPD1; LMNA; CALML5; HPSE; TMED9; ACPP; FUCA1; HEBP2; S100A11; RPS27A; IL1RN; CKMT1B; CTSD; TECR; KRT10; HEXB; BLMH; CAT; SYLP1; AGA; CTSH; STS; C11orf54; RNASE7; IL37; KPRP; KRT6B; KRT75; NRM; LGALS3BP; APOE; FAM175B; CDA; KRT76; KRTAP13-2; and CRYAB;
(b) identifying the levels of the subset of one or more biomarker proteins in step (a) relative to levels of the same proteins in a skin sample from control subjects; wherein increased levels of the subset of one or more biomarker proteins in the subject's sample relative to control levels identifies aging skin in the subject; and
(c) administering to the subject identified as having aging skin in step (b) a treatment that decreases the levels of the subset of one or more biomarker proteins in the subject's skin, thereby treating aging skin in the subject.

10. A kit for determining skin aging or skin attribute status in a human subject, the kit comprising:
(a) a substrate for collecting a skin sample from the subject; and
(b) reagents for quantifying the levels of one or more biomarker proteins relative to control levels, wherein said biomarker protein is selected from the group consisting of: GLUD1; DDX3X; ERGIC1; NIT2; PSMC5; TSG101; UQCRFS1; ZG16B; RAB1A; PLA2G4E; GPD2; AADAC; ATP6V1B2; PHB; NAPRT; RAB1B; SULT2B1; CARS; HSPA9; ACTR2; EIF2S1; RPL22; CAP1; ATP6V1E1; RTN3; DYNLL2; PHB2; ITPA; IST1; UQCRC2; FASN; LMNB2; MY018A; ATP6V1A; ACOX1; RPN2; RAB7A; EIF4A1; ALDH2; ACTR3; ACADVL; KRT73; ANXA5; HNRNPH2; VCP; MYH14; ANXA4; PDCD6; HSPE1; IDE; CKAP4; PIP; CAPZB; ASPRV1; MT-CO2; CAPG; RAB14; PTGS1; POF1B; HSPA5; TGM5; HSPA8; LGALS7B; HSPD1; LMNA; CALML5; HPSE; TMED9; ACPP; FUCA1; HEBP2; S100A11; RPS27A; IL1RN; CKMT1B; CTSD; TECR; KRT10; HEXB; BLMH; CAT; SYLP1; AGA; CTSH; STS; C11orf54; RNASE7; IL37; KPRP; KRT6B; KRT75; NRM; LGALS3BP; APOE; FAM175B; CDA; KRT76; KRTAP13-2; and CRYAB.

11. The kit according to claim 10 further comprising:
(c) reagents for analyzing and/or quantifying the levels of the one or more extracted biomarker proteins from the subject relative to control levels.

12. The kit of claim 10, comprising reagents for analyzing and/or quantifying the levels of a subset of biomarker proteins selected from the group consisting of GLUD1, DDX3X, ERGIC1, NIT2, PSMC5, TSG101, UQCRFS1, ZG16B, RAB1A, PLA2G4E, GPD2, AADAC, ATP6V1B2, PHB, NAPRT, RAB1B, SULT2B1, CARS, HSPA9, ACTR2, EIF2S1, RPL22, CAP1, ATP6V1E1, RTN3, DYNLL2, PHB2, ITPA, IST1, UQCRC2, FASN, LMNB2, MYO18A, ATP6V1A, ACOX1, RPN2, RAB7A, EIF4A1, ALDH2, ACTR3, ACADVL, KRT73, ANXA5, HNRNPH2, VCP, MYH14, ANXA4, PDCD6, HSPE1, IDE, CKAP4, PIP, CAPZβ, ASPRV1, MT-CO2, CAPG, RAB14, PTGS1, POF1B, HSPA5; TGM5, HSPA8, LGALS7B, HSPD1, CALML5, HPSE, TMED9, ACPP, FUCA1, HEBP2, S100A11, RPS27A, IL1RN, CKMT1B and CTSD.

13. The kit of claim 10, further comprising reagents for analyzing and/or quantifying the levels of a subset of biomarker proteins selected from the group consisting of HSPA9, HSPA5, HSPA8 and a combination thereof.

14. The kit of claim 10, comprising reagents for analyzing and/or quantifying the levels of a subset of biomarker proteins selected from the group consisting of TECR, KRT10, HEXB, BLMH, CAT, SYPL1, AGA, CTSH, STS, C11orf54, RNASE7, IL37, KPRP, KRT6B, KRT75, NRM, LGALS3BP, APOE, FAM175B; CDA, KRT76, KRTAP13-2 and CRYAB.

15. The method of claim 1, wherein said skin biomarker protein is selected from the group consisting of HSPA9, HSPA5, HSPA8 and a combination thereof.

16. The method of claim 8, wherein said biomarker protein is selected from the group consisting of HSPA9, HSPA5, HSPA8 and a combination thereof.

17. The method of claim 9, wherein said biomarker protein is selected from the group consisting of HSPA9, HSPA5, HSPA8 and a combination thereof.
